(19) United States
(12) Patent Application Publication
(10) Pub. No.: US 2023/0380942 A1
(43) Pub. Date: Nov. 30, 2023

(54) INTRAORAL SCANNER WITH WAVEGUIDE PATTERN PROJECTOR

(71) Applicant: Align Technology, Inc., San Jose, CA (US)

(72) Inventors: Nadav Fain, Petach Tikva (IL); Eliahou Franklin Nizard, Jerusalem (IL)

(21) Appl. No.: 18/142,548

(22) Filed: May 2, 2023

Related U.S. Application Data
(60) Provisional application No. 63/346,272, filed on May 26, 2022.

(51) Int. Cl. A61C 9/00; A61B 1/00; A61B 1/24

(52) U.S. Cl. CPC A61C 9/006; A61B 1/00096; A61B 1/24; A61B 1/00097

(57) ABSTRACT

An intraoral scanner comprises an elongate wand comprising a probe at a distal end of the elongate wand, one or more light sources disposed within the elongate wand away from the distal end of the probe, one or more waveguides disposed within the probe, and one or more image sensors disposed at the distal end of the probe. The one or more light sources are configured to generate unpatterned light, The one or more waveguides are configured to receive the unpatterned light generated by the one or more light sources and to output patterned light from one or more locations at the distal end of the probe. The one or more image sensors are configured to capture images of intraoral objects illuminated by the patterned light.

### RELATED APPLICATIONS

The present application claims the benefit under 35 U.S.C. § 119(e) of U.S. Provisional Patent Application No. 63/346,272, filed May 26, 2022, which is incorporated by reference herein.

### TECHNICAL FIELD

Embodiments of the present disclosure relate to intraoral scanners that use structured light projection and, in particular, to an intraoral scanner that includes a waveguide pattern projector.

### BACKGROUND

A great variety of methods and systems have been developed for direct optical measurement of teeth and the subsequent automatic manufacture of orthodontics and prosthodontics. The term “direct optical measurement” signifies surveying of teeth in the oral cavity of a patient. This facilitates the digital construction of virtual three-dimensional (3D) models of dental arches, which may be used for the computer-assisted design (CAD) or computer-assisted manufacture (CAM) of prosthetics and/or orthodontics without having to make any cast impressions of the patient's teeth. Such systems typically include an optical probe that is inserted into a patient's mouth, where the probe includes a means for generating intraoral scans.

One type of system that performs intraoral scanning is a system that uses a projected light pattern to image a three dimensional surface. Such systems that use a projected light pattern typically include one or more image sensors and one or more pattern projectors disposed at a distal end of the probe (e.g., the end that gets inserted into the patient's mouth). However, by including the image sensors and light projectors at the distal end of the probe, the thickness of the probe is increased. Such increased thickness can reduce a patient comfort when the probe is inserted into the patient's mouth. Additionally, such increased thickness can reduce an ability of the doctor using the intraoral scanner to properly position the probe for optimal scan acquisition. Moreover, intraoral scanners with cameras and structured light projectors in the distal end of the probe can suffer from heat buildup at the distal end of the probe.

### SUMMARY

Some example implementations are summarized herein.

In a first implementation an intraoral scanner comprises: an elongate wand comprising a probe at a distal end of the elongate wand; one or more light sources disposed within the elongate wand away from the distal end of the probe, wherein the one or more light sources are configured to generate unpatterned light; one or more waveguides disposed within the probe, wherein the one or more waveguides are configured to receive the unpatterned light generated by the one or more light sources and to output patterned light from one or more locations at the distal end of the probe; and one or more image sensors disposed at the distal end of the probe, wherein the one or more image sensors are configured to capture images of intraoral objects illuminated by the patterned light.

A second implementation may extend the first implementation. In the second implementation, the one or more waveguides comprise one or more pattern projectors at the distal end of the probe, wherein each pattern projector outputs at least a portion of the patterned light.

A third implementation may extend the second implementation. In the third implementation, each of the one or more pattern projectors comprises a focusing grating coupler.

A fourth implementation may extend the third implementation. In the fourth implementation, at least one focusing grating coupler is configured to output a pattern of spots.

A fifth implementation may extend the fourth implementation. In the fifth implementation, the pattern of spots comprises four to sixteen spots having a uniform spacing.

A sixth implementation may extend the any of the second through fifth implementations. In the sixth implementation, each waveguide of the one or more waveguides comprises a transport region having a first width, a pattern projector region comprising the pattern projector, the pattern projector region having a second width that is greater than the first width, and a tapered region between the transport region and the pattern projector region that transitions from the first width to the second width.

A seventh implementation may extend the any of the second through sixth implementations. In the seventh implementation, the intraoral scanner further comprises a microlens array disposed over each of the one or more pattern projectors.

An eighth implementation may extend the any of the first through seventh implementations. In the eighth implementation, the one or more waveguides comprise at least one waveguide configured to perform the following: receive the unpatterned light from a light source of the one or more light sources; and divide the unpatterned light across a plurality of paths, wherein each path of the plurality of paths comprises a distinct pattern generator configured to output a portion of the patterned light.

A ninth implementation may extend the eighth implementation. In the ninth implementation, each distinct pattern generator comprises a focusing grating coupler.

A 10^{th} implementation may extend the any of the first through ninth implementations. In the 10^{th} implementation, the probe comprises a longitudinal axis, wherein the one or more image sensors each have an angle of 45 degrees to 135 degrees to the longitudinal axis, and wherein the patterned light is projected from the one or more locations at the distal end of the probe at one or more angles of 45 degrees to 135 degrees to the longitudinal axis.

An 11^{th} implementation may extend the any of the first through 10^{th} implementations. In the 11th implementation, a lateral distance between the one or more locations at the distal end of the probe and the one or more image sensors within a plane defined at least in part by the longitudinal axis of the probe is less than 2 mm.

A 12^{th} implementation may extend the any of the first through 11^{th} implementations. In the 12th implementation, the patterned light has an approximately constant overlap over an entire depth of focus of the intraoral scanner.

A 13^{th} implementation may extend the any of the first through 12^{th} implementations. In the 13th implementation, all light rays of the patterned light output from a particular location of the one or more locations are approximately parallel to one another.

A 14^{th} implementation may extend the 13^{th} implementation. In the 14^{th} implementation, first light rays of the patterned light output from a first location of the one or more locations have a first angle relative to a plane defined at least in part by a longitudinal axis of the probe, and wherein second light rays of the patterned light output from a second location of the one or more locations have a second angle relative to the plane defined at least in part by the longitudinal axis.

A 15^{th} implementation may extend the any of the first through 14^{th} implementations. In the 15th implementation, the one or more light sources comprise a first light source that outputs first coherent light having a first wavelength and a second light source that outputs second coherent light having a second wavelength, wherein a same waveguide of the one or more waveguides is to transport the first coherent light and the second coherent light to the one or more locations at the distal end of the probe.

A 16^{th} implementation may extend the any of the first through 15^{th} implementations. In the 16th implementation, a) the intraoral scanner further comprises at least one of a lens, a prism or a grating that couples a light source of the one or more light sources to a waveguide of the one or more waveguides or b) the light source directly couples to the waveguide.

A 17^{th} implementation may extend the any of the first through 16^{th} implementations. In the 17th implementation, the one or more waveguides comprises a first plurality of waveguides, and the one or more light sources comprises a plurality of semiconductor lasers.

An 18^{th} implementation may extend the any of the first through 17^{th} implementations. In the 18th implementation, the intraoral scanner further comprises: a refractive coating on at least one surface of the one or more waveguides at the one or more locations.

A 19^{th} implementation may extend the any of the first through 18^{th} implementations. In the 19th implementation, the intraoral scanner further comprises a first plate in the probe, wherein the first plate covers at least a first subset of the one or more image sensors such that returning light from the imaged intraoral objects passes through the first plate to reach the first subset of the one or more image sensors, wherein the first plate comprises at least a first subset of the one or more waveguides.

A 20^{th} implementation may extend the 19^{th} implementation. In the 20^{th} implementation, the intraoral scanner further comprises a second plate in the probe disposed on the first plate such that the second plate also covers at least the first subset of the one or more image sensors, wherein the second plate comprises at least a second subset of the one or more waveguides, wherein the first subset of the one or more waveguides output the patterned light from a first subset of the one or more locations, and wherein the second subset of the one or more waveguides output the patterned light from a second subset of the one or more locations that do not overlap with the first subset of the one or more locations.

A 21^{st} implementation may extend the 19^{th} implementation. In the 21^{st} implementation, the first plate has a first orientation relative to a plane defined at least in part by a longitudinal axis of the probe, and the intraoral scanner further comprises a second plate in the probe, wherein the second plate covers at least a second subset of the one or more image sensors such that returning light from the imaged intraoral objects passes through the second plate to reach the second subset of the one or more image sensors, wherein the second plate comprises at least a second subset of the one or more waveguides, and wherein the second plate has a second orientation relative to the plane defined at least in part by the longitudinal axis of the probe.

In a 22^{nd} implementation, an intraoral scanner comprises: an elongate wand comprising a probe at a distal end of the elongate wand; one or more light sources disposed within the elongate wand, wherein the one or more light sources are configured to generate unpatterned light, and wherein the one or more light sources are oriented to project the unpatterned light in a first direction along a length of the elongate wand; and one or more pattern generating optical elements disposed within the probe, wherein the one or more pattern generating optical elements are configured to receive the unpatterned light generated by the one or more light sources and to output patterned light from one or more locations of the probe.

A 23^{rd} implementation may extend the 22^{nd} implementation. In the 23^{rd} implementation, the one or more pattern generating optical elements comprise one or more waveguides.

A 24^{th} implementation may extend the 23^{rd} implementation. In the 24^{th} implementation, each of the one or more waveguides comprises one or more focusing grating couplers that generates the patterned light at the one or more locations.

A 25^{th} implementation may extend the 22^{nd} through 24^{th} implementations. In the 25^{th} implementation, the one or more locations are at the distal end of the probe.

A 26^{th} implementation may extend the 22^{nd} through 25^{th} implementations. In the 26^{th} implementation, the one or more light sources are located away from the distal end of the probe, and the one or more pattern generating optical elements are to output the patterned light in a second direction that is at an angle to the first direction and in a field of view of one or more cameras of the intraoral scanner.

A 27^{th} implementation may extend the 22^{nd} through 26^{th} implementations. In the 27^{th} implementation, the one or more pattern generating optical elements comprises a reflector to redirect the unpatterned light from the first direction to the second direction.

In a 28^{th} implementation, an intraoral scanner comprises: one or more lasers disposed within the intraoral scanner away from a distal end of the intraoral scanner, wherein the one or more lasers are configured to generate first unpatterned light having a first wavelength; one or more waveguides disposed within the probe, wherein the one or more waveguides each comprise a transport region configured to receive the first unpatterned light and transport the unpatterned light to a projection region comprising a focusing grating coupler, wherein the focusing grating coupler is to output patterned light onto an object external to the intraoral scanner; and one or more image sensors disposed at the distal end of the intraoral scanner, wherein the one or more image sensors are configured to capture images of intraoral objects illuminated by the patterned light.

### BRIEF DESCRIPTION OF THE DRAWINGS

Embodiments of the present disclosure are illustrated by way of example, and not by way of limitation, in the figures of the accompanying drawings.

FIG. 1 illustrates an intraoral scanner having image sensors disposed at a distal end of the intraoral scanner and one or more light sources disposed away from the distal end of the intraoral scanner, in accordance with an embodiment.

FIG. 2A illustrates a probe of an intraoral scanner and shows a first field of projection of patterned light output be the intraoral scanner, in accordance with an embodiment.

FIG. 2B illustrates a probe of an intraoral scanner and shows a second field of projection of patterned light output be the intraoral scanner, in accordance with an embodiment.

FIG. 3A illustrates a first waveguide configuration for use in an intraoral scanner, in accordance with an embodiment.

FIG. 3B illustrates a second waveguide configuration for use in an intraoral scanner, in accordance with an embodiment.

FIG. 3C illustrates a third waveguide configuration for use in an intraoral scanner, in accordance with an embodiment.

FIG. 4A illustrates an example layout of a camera, a waveguide and a light source in an intraoral scanner, in accordance with an embodiment.

FIG. 4B illustrates an example layout of a set of cameras and a set of waveguides in an intraoral scanner, in accordance with an embodiment.

FIG. 4C is a chart depicting a plurality of different configurations for the position of cameras in a probe of an intraoral scanner, in accordance with some embodiments of the present disclosure.

FIG. 5A illustrates a perspective view of an example waveguide that includes a focusing grating coupler coupled to a laser via a coupling, in accordance with an embodiment.

FIG. 5B illustrates a perspective view of an example waveguide, that includes a focusing grating coupler, directly coupled to a laser, in accordance with an embodiment.

FIG. 6 illustrates a top view of a focusing grating coupler for use in a waveguide, in accordance with an embodiment.

FIG. 7A illustrates a perspective view of an example waveguide that includes a focusing grating coupler configured to output patterned light having a first focused spot pattern, in accordance with an embodiment.

FIG. 7B illustrates a perspective view of an example waveguide that includes a focusing grating coupler configured to output patterned light having a second focused spot pattern, in accordance with an embodiment.

FIG. 7C illustrates an FGC region divided into a grid of 10×10 squares, in accordance with one embodiment.

FIG. 8 illustrates a top view of a focusing grating coupler design that outputs a spot pattern, in accordance with an embodiment.

FIG. 9 illustrates a perspective view of an example waveguide that includes a focusing grating coupler configured to output patterned light having a focused spot pattern and a microlens array over the focusing grating coupler, in accordance with an embodiment.

FIGS. 10A-B illustrate views of a focus grating coupler that show how to design a focus grating coupler for use in an intraoral scanner, in accordance with an embodiment.

### DETAILED DESCRIPTION

Described herein are embodiments of an intraoral scanner that uses structured light (also referred to as patterned light) projection to determine depth information of imaged objects (e.g., of imaged intraoral objects that are in an oral cavity of a patient). There are multiple intraoral scanners that use the principle of structured light projection to generate three-dimensional (3D) scans of intraoral objects. One example of such an intraoral scanner that uses structured light projection s described in U.S. Patent Publication No. 2019/0388193, filed Jun. 19, 2019, entitled “Intraoral 3D Scanner Employing Multiple Miniature Cameras and Multiple Miniature Pattern Projectors,” which is incorporated by reference herein in its entirety. Another example of such an intraoral scanner that uses structured light projection is described in U.S. Patent Publication No. 2020/0404243, filed Jun. 23, 2020, also entitled “Intraoral 3D Scanner Employing Multiple Miniature Cameras and Multiple Miniature Pattern Projectors,” which is incorporated by reference herein in its entirety. Such existing intraoral scanners that use structured light projection generally rely on structured light projectors that are disposed at a distal end of a probe of the intraoral scanner that gets inserted into a patient's mouth. Such structured light projectors have a minimum size, which imposes a constraint on the minimum thickness of the intraoral scanner's probe.

It is generally desirable for probes of intraoral scanners to be as thin as possible. Thinner intraoral scanner probes are more easily inserted into patient oral cavities, and cause less discomfort to the patients. Moreover, thinner intraoral scanner probes have more room to maneuver within the oral cavity due to their decreased thickness. However, the size of structured light projectors that are disposed in the distal end of intraoral scanner probes cause those probes to be relatively thick. Thus, traditional intraoral scanners that use structured light projection have historically had a minimum probe thickness that has been necessary to accommodate the size of the structured light projectors.

Additionally, components such as structured light projectors and cameras in the distal end of the intraoral scanner probe each generate some amount of heat. Having all of these components in the distal end of the probe can cause heat buildup to occur in the distal end of the probe. Accordingly, one or more cooling techniques may be necessary to mediate such heat buildup. Thus, structured light projectors may require heat flow systems, electrical contacts, and so on, which may contribute to a thickness of the intraoral scanner's probe.

In embodiments, one or more light sources are disposed away from the distal end of the probe. The one or more light sources may be oriented to direct light in a first direction along a length of an elongate wand (e.g., approximately along a longitudinal axis of the elongate wand) comprising the probe. The light emitted by the one or more light sources may be unpatterned light, which may be directed generally along a longitudinal axis of the elongate wand to a proximal end of the probe, where the light may enter a pattern generating optical element that may output patterned light. The patterned light may be output in a second direction that is at an angle to the first direction, and may be in a field of view of one or more cameras of the intraoral scanner. In some embodiments, the pattern generating optical element includes a reflector such as a mirror or prism that redirects the light from the first direction to the second direction. In some embodiments, the one or more light sources are coupled to waveguides that transport generated light to the distal end of the probe. Alternatively, the light may be emitted towards the distal end of the probe without use of waveguides. The waveguides may be thin sheets of material that are transparent to a wavelength of light that is used. Examples of materials for waveguides include doped glass, silicon, doped SiO₂, GaAs, AsInP, Si₃N₄ (silicon nitride), or silica. The light sources may be configured to generate unstructured or unpatterned light, and the waveguides may have a patterned region (also referred to as a pattern projector) that emits a patterned or structured light. In some embodiments, the waveguides or the patterned region of the waveguides may be referred to as a pattern generating optical element. The patterned regions may be pattern projectors that have a significantly smaller footprint than traditional structured light projectors. The use of waveguides with pattern projectors for producing patterned light reduce a size (e.g., a thickness and/or width) of a probe and allow for additional degrees of freedom of spatial design of the intraoral scanner. In embodiments, the patterned region includes a focusing grating coupler (FGC) that outputs patterned light having a preconfigured pattern. Since the light sources are not disposed in the probe at the distal end of the probe, the probe (or at least the distal end of the probe) may be significantly thinner than has heretofore been achievable. Additionally, heat buildup at the distal end of the probe may be reduced by disposing the light sources away from the distal end of the probe.

In some embodiments, the patterned region of one or more waveguides may be configured to generate unpatterned light, such as a focused ray. Embodiments are discussed with regards to waveguides having a patterned region that generates patterned light. However, it should be understood that all such embodiments discussed herein may alternatively or additionally include waveguides having patterned regions that generate unpatterned light. Additionally, it should be understood that embodiments also cover use of pattern generating optical elements that do not include waveguides or focusing grating couplers. For example, a pattern generating optical element may include a reflector (e.g., a mirror or prism) that redirects light from a first direction to a second direction and a diffractive optical element (DOE) that receives the light having the second direction and outputs patterned light.

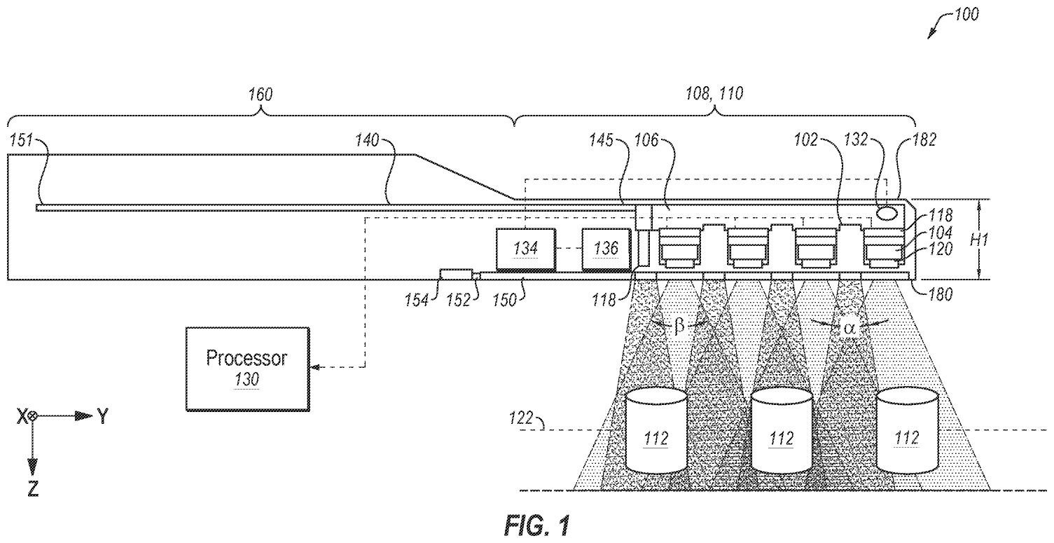

Reference is now made to FIG. 1, which is a schematic illustration of an intraoral scanner 100 comprising an elongate wand (e.g., a body with a probe 108 at one end of the body) such as an elongate handheld wand, in accordance with some applications of the present disclosure. The intraoral scanner 100 may be a wireless scanner that includes a wireless module (not shown) disposed in a body of the intraoral scanner 100 for wirelessly connecting to a computing device. Alternatively, the intraoral scanner 100 may be a wired scanner that connects to a computing device via a wired connection. Alternatively, the intraoral scanner 100 may be configured to operate in a wired mode or a wireless mode, depending on circumstances.

Intraoral scanner 100 includes one or more light sources 154 optically coupled to one or more waveguides in one or more substrates 150 (e.g., directly or via optical couplers 152 such as lenses, prisms and/or gratings). In one embodiment, light source(s) 154 optically couple to one or more waveguides directly (e.g., by direct connection between a laser die or optical fiber and an edge of a waveguide). In some embodiments, an edge of the waveguide may be tapered (e.g., horizontal width of the waveguide may grow smaller towards the edge). A tapered edge of the waveguide may provide edge coupling between the waveguide and the one or more light sources 150. In one embodiment, the optical coupler 152 is a grating constructed on top of or etched into the waveguide. As shown, the light source(s) 154 are not disposed at the distal end of the probe (i.e., are disposed away from the distal end of the probe) in some embodiments. The light source(s) 154 may be disposed, for example near a center of the intraoral scanner 100, at a proximal end of the intraoral scanner 100, at a proximal end of the probe 108, or at another location within intraoral scanner 100 that is away from the distal end of the intraoral scanner 100. As shown, the light source(s) 154 may be positioned and oriented to output light that is approximately aligned with a longitudinal axis of the intraoral scanner 100 (e.g., along the Y-axis in FIG. 1).

In some embodiments, one or more light sources 154 are disposed at or near the distal end of the probe 108, but have an orientation that is approximately parallel to a longitudinal axis of the intraoral scanner 100 (or to the probe 108 of the intraoral scanner, such as if the probe has a longitudinal axis that is different from a longitudinal axis of a remainder of the elongate handheld wand). By orienting the light sources 154 so that they project light approximately parallel to the longitudinal axis (e.g., along the Y axis) of the intraoral scanner probe 108 and/or so that they are away from a distal end of the probe 108, a thickness (H1) of the probe 108 can be minimized.

In embodiments, the one or more light sources 154 are lasers (e.g., integrated semiconductor laser projectors) configured to output one or more wavelengths of coherent light. For example, light sources 154 may include one or more first light sources that output light having a first wavelength (e.g., blue light) and one or more second light sources that output light having a second wavelength (e.g., green light). The light output by the light sources 154 may be unpatterned light. Accordingly, the light entering the waveguides in the substrates 150 may be unpatterned light. However, the light exiting the waveguides in the substrates 150 in embodiments is patterned light.

The one or more waveguides are formed in thin substrates 150 (e.g., sheets/plates of glass or silica) with one or more doped or deposited regions that form the waveguides and that transport light from the light source(s) 154. In one embodiment, the waveguide(s) are formed in substrates 150 such as fused silica substrates, glass substrates, plastic substrates, etc. that include one or more regions having an increased index of refraction as compared to surrounding regions of the substrate. The regions with an increased index of refraction may achieve the increased index of refraction via the introduction of one or more additional materials, e.g., by creating doped regions via doping the substrate with molecules of another material, by depositing another material on top of or within the substrate at the regions (e.g., via chemical vapor deposition (CVD), and so on. The one or more regions may be channels (also referred to as transport regions) within the substrate(s) 150 having dopants or other additional molecules that increase a refractive index at the regions. In some embodiments, the waveguide may have a width (i.e., lateral width) of about 1-5 microns. In some embodiments, the waveguide may have a lateral width that is a width of the substrate. In such an instance, the waveguide may not be a channel, but may instead be a thin film, and light may be bound only in the z-axis. Accordingly, the light may diverge in the x-axis as it propagates in the y-axis (e.g., similar to a ray of light in free space). In such an embodiment, a taper section (discussed below) may be omitted. In embodiments, the channels of the waveguide(s) may have a depth of about 1-5 microns in the substrate. In embodiments, the sheet/plate that includes the one or more waveguides has a thickness of about 0.5-2 mm (e.g., about 1 mm).

In one embodiment, as shown, the substrate (or substrates) 150 comprising the waveguide(s) are positioned on the probe such that they are between the cameras 104 and the object 112 being imaged. Accordingly, light reflecting off of the object 112 may pass through the substrate(s) 150 comprising the waveguide(s) to reach the cameras 104. Alternatively, the substrate 150 comprising the waveguide(s) may be in a same plane as the cameras 104. In such an embodiment, the substrate may include cutouts for the cameras 104. In another embodiment, the substrate 150 comprising the waveguide(s) is positioned behind the cameras. In such an embodiment, light does not pass through the substrate to reach the cameras. In one embodiment, the substrate(s) 150 comprising the waveguide(s) forms a window of the intraoral scanner 100.

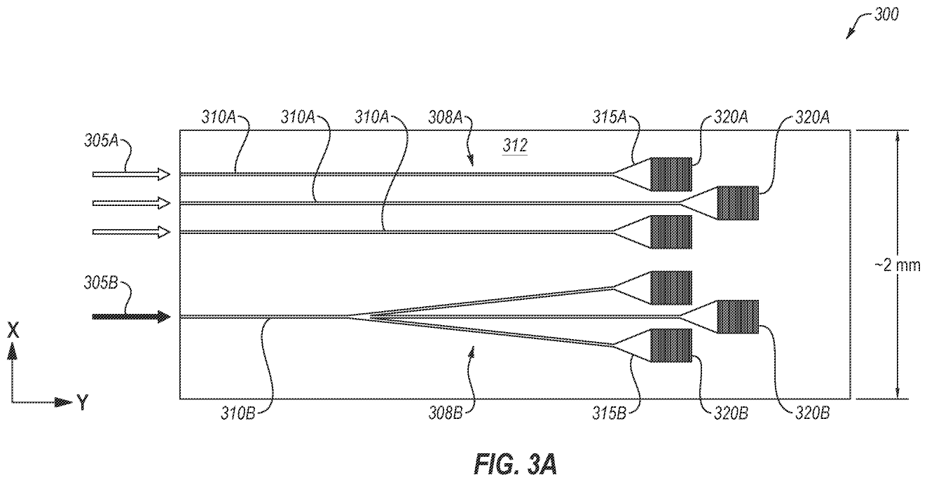

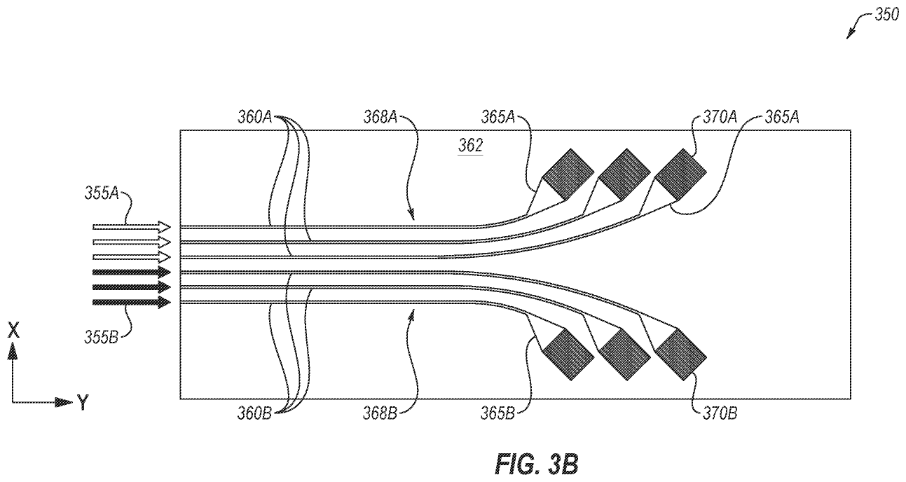

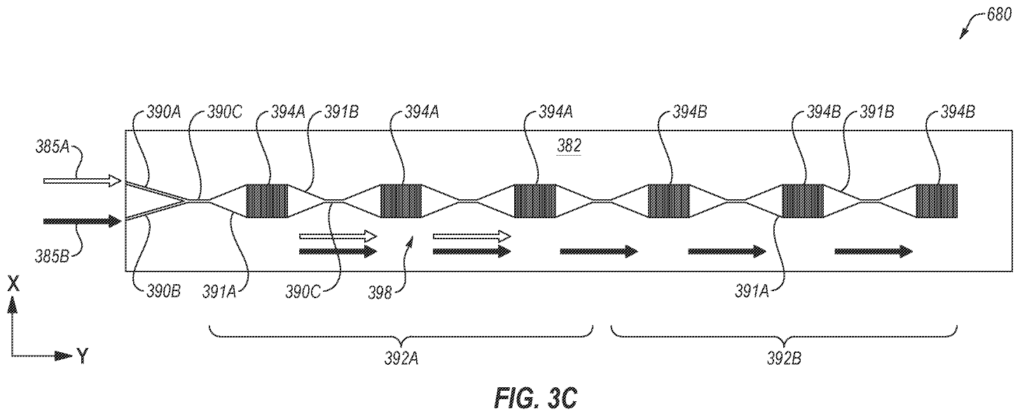

The waveguide(s) may each include one or more pattern projectors 102 (also referred to as projection regions) that are configured to emit patterned or structured light from the waveguide(s) at one or more locations at (or near) the distal end of the probe 108. In embodiments, each of the one or more pattern projectors 102 is a focusing grating coupler (FGC) that has been configured to output a particular pattern of light (or in some instances unpatterned light). An FGC may include an array of grooves, lines, and/or other features that are etched into and/or otherwise formed in, on or under the waveguide (e.g., into a plate of doped silica). The grooves/lines/features may be curved and chirped, such that their average period is of the scale of a wavelength of the light that will be emitted into the waveguide. The grooves/lines/features may be formed using conventional fabrication methods, such as using e-beam lithography, doping, deposition, and/or nano-imprinting. For example, a photoresist may be formed on a substrate, and the photoresist may then be patterned and selectively cured using lithography or nano-imprinting. Cured or uncured portions of the photoresist may then be removed. Exposed regions of the substrate that are not covered by photoresist may be etched, and then a remainder of the photoresist may be removed after the etching. In some embodiments, the FGC includes an array of doped regions in the substrate that have a different refractive index than a refractive index of the waveguide (e.g., of the tapered region or transport region of the waveguide). Any of the FGCs described in embodiments (e.g., as shown in FIGS. 3A-3C and 5-10B) may include an array of grooves, an array of deposited features/lines, or an array of regions of increased or decreased refractive index (e.g., formed via doping) as compared to a remainder of the waveguide. In some embodiments, the array of grooves, lines, features and/or doped regions (e.g., with increased or decreased refractive index) may be fabricated on top of a waveguide (e.g., on top of a projection region of a waveguide), on a bottom of a waveguide (e.g., at an interface of the waveguide with an undoped portion of the substrate containing the waveguide), or on a top and bottom of the waveguide.

As shown, light that travels through the pattern projectors 102 (e.g., through the FGCs) is emitted from the pattern projectors 102 at an angle to the longitudinal axis (e.g., approximately along the z-axis, which is orthogonal to the longitudinal axis). The emitted patterned light may be output at an angle of about, for example, 45 degrees to 135 degrees to the longitudinal axis (e.g., to the y-axis), and thus may be projected from the one or more locations of the pattern projectors 102 at the distal end of the probe 108 at one or more angles of 45 degrees to 135 degrees to the longitudinal axis. The emitted patterned light may additionally or alternatively have an angle of about 45 degrees to about 135 degrees to the x-axis shown in FIG. 1. Different pattern projectors 102 may be configured to emit light at different angles. Accordingly, first light rays of the patterned light output from a first location may have a first angle relative to a plane defined at least in part by a longitudinal axis of the probe, and second light rays of the patterned light output from a second location may have a second angle relative to the plane defined at least in part by the longitudinal axis. The pattern projectors 102 may be configured in embodiments to radiate any target beam shape. Examples of such beam shapes include a pattern of spots (e.g., four to sixteen spots, twelve spots, nine spots, etc.), a pattern of lines, a grid pattern, a checkerboard pattern, and so on.

Use of the waveguides with integrated pattern projectors (e.g., FGCs) enables the height (i.e., thickness) H1 of the probe 28 to be minimized. In some applications, a height H1 of probe 108 is less than 15 mm, or less than 10 mm, or less than 8 mm, or less than 6 mm, with height H1 of probe 108 being measured from a lower surface (sensing surface) 180, through which reflected light from object 112 being scanned enters probe 108, to an upper surface 182 opposite lower surface 180. In some applications, the height H1 is between 4-10 mm.

In some embodiments, the light beams output by a particular pattern projector 102 are approximately parallel to one another. Accordingly, the light pattern may remain focused within a wide focal range of the intraoral scanner 100 (e.g., over a range of a few mm). As opposed to designs in which patterned light projectors are disposed at the distal end of the probe and pointed towards object 112, use of pattern projectors 102 (e.g., FGCs) in or at the window of the probe enables a much wider latitude for design with regards to an angle and field of projection for projected patterns. For example, because the number of locations from which patterned light is emitted can be greatly increased using the pattern projectors 102 described herein, the field of illumination a (alpha) of the individual pattern projectors 102 may be below 70 degrees.

In embodiments, the patterned light output by a pattern projector 102 is two-dimensional (2D) fan of focused beams (e.g., that may form a pattern of projected spots over a focal range of the intraoral scanner 100).

Many waveguides and/or pattern projectors 102 may be used in the intraoral scanner 100 to design any desired pattern of light. The waveguides and integrated pattern projectors 102 are very small, and so many such waveguides/pattern projectors may fit into the head of a single probe (e.g., the distal end of the probe 108).

In embodiments, the probe may include first waveguides configured to transport light having a first wavelength and second waveguides configured to transport light having a second wavelength. In embodiments, different wavelengths of light may be output by the same waveguides (e.g., pattern projectors 102 of waveguides in substrate 150). Different wavelengths of light output by a same FGC may have a different pattern, angle, focus properties, and so on. In embodiments, different wavelengths of light are output by a same waveguide and pattern projector serially (e.g., one after the other). Alternatively, multiple wavelengths of light may be output by a same waveguide and pattern projector in parallel (e.g., simultaneously).

Use of waveguides with integrated pattern projectors 102 and light sources 154 that are away from a distal end of probe 108 enables a radiating system of intraoral scanner 100 to be integrated into a single silica window having a width of about 12 mm down to about 1 mm in embodiments.

In some embodiments, a first plate including a first set of waveguides is disposed in the distal end of the probe 108, wherein the first plate covers at least a first subset of the one or more image sensors such that returning light from the imaged intraoral objects passes through the first plate to reach the first subset of the one or more image sensors. In some embodiments, a second plate is disposed in the probe on the first plate such that the second plate also covers at least the first subset of the one or more image sensors, wherein the second plate comprises a second set of waveguides. The first set of waveguides output the patterned light from a first set of one or more locations, and the second set of waveguides output the patterned light from a second set of one or more locations that do not overlap with the first subset of the one or more locations. In some embodiments, a second plate in the probe is disposed on the first plate such that the second plate also covers at least the first set of one or more image sensors, wherein the second plate comprises a second set of waveguides, wherein the first set of waveguides output the patterned light from a first subset of the one or more locations, and wherein the second set of waveguides output the patterned light from a second set of the one or more locations that do not overlap with the first subset of the one or more locations.

In some embodiments, the first plate has a first orientation relative to a plane defined at least in part by a longitudinal axis of the probe 108. The probe 108 further includes a second plate in the probe that covers at least a second subset of the one or more image sensors such that returning light from the imaged intraoral objects passes through the second plate to reach the second subset of the one or more image sensors. The second plate comprises a second set of waveguides, and the second plate has a second orientation relative to the plane defined at least in part by the longitudinal axis of the probe, which may be different from the first orientation.

In embodiments, a plurality of cameras 104 are disposed at or near a distal end of probe 108. The cameras 104 may be coupled to a rigid structure 106 disposed within probe 108 at a distal end 110 of the body of the intraoral scanner 100. In some applications, during an intraoral scanning procedure, probe 108 is inserted into the oral cavity of a subject or patient.

For some applications, the pattern projectors 102 of the waveguides in substrate 150 are configured such that patterned light emitted by the pattern projectors 102 projects onto an object 112 outside of intraoral scanner 100 that is placed in its field of illumination. Cameras 104 may be positioned within probe 108 such that each camera 104 faces an object 112 outside of intraoral scanner 100 that is placed in its field of view. Alternatively, one or more image sensors may be positioned in a proximal end of the intraoral scanner and the object 112 may be viewed by reflection of light off a mirror and into the image sensor. The positioning and configuration of pattern projectors 102 and the cameras 104 within probe 108 as described herein enables the intraoral scanner 100 to have an overall large field of view while maintaining a low profile (e.g., a small thickness H1).

In some applications, cameras 104 each have a large field of view β (beta) of at least 45 degrees, e.g., at least 70 degrees, e.g., at least 80 degrees, e.g., 85 degrees. In some applications, the field of view may be less than 120 degrees, e.g., less than 100 degrees, e.g., less than 90 degrees. In one embodiment, a field of view β (beta) for each image sensor is between 80 and 90 degrees, which may be particularly useful because it provided a good balance among pixel size, field of view and camera overlap, optical quality, and cost. Cameras 104 may include an image sensor 118 and objective optics 120 including one or more lenses. To enable close focus imaging, cameras 104 may focus at an object focal plane 122 that is located between 1 mm and 30 mm, e.g., between 4 mm and 24 mm, e.g., between 5 mm and 11 mm, e.g., 9 mm -10 mm, from the lens that is farthest from the sensor. In some applications, cameras 104 may capture images at a frame rate of at least 30 frames per second, e.g., at a frame of at least 75 frames per second, e.g., at least 100 frames per second. In some applications, the frame rate may be less than 200 frames per second.

A large field of view achieved by combining the respective fields of view of all the cameras may improve accuracy due to reduced amount of image stitching errors, especially in edentulous regions, where an imaged gum surface is smooth and there may be fewer clear high resolution 3D features. Having a larger field of view enables large smooth features, such as the overall curve of a tooth, to appear in each image frame, which improves the accuracy of stitching respective surfaces obtained from multiple such image frames.

For some applications, in order to improve image capture, each camera 104 has a plurality of discrete preset focus positions, in each focus position the camera focuses at a respective object focal plane 122. Each of cameras 104 may include an autofocus actuator that selects a focus position from the discrete preset focus positions in order to improve a given image capture. Additionally or alternatively, each camera 104 includes an optical aperture phase mask that extends a depth of focus of the camera, such that images formed by each camera are maintained focused over all object distances located between 1 mm and 30 mm, e.g., between 4 mm and 24 mm, e.g., between 5 mm and 11 mm, e.g., 9 mm -10 mm, from the lens that is farthest from the sensor.

In some applications, cameras 104 and pattern projectors 102 (e.g., FGCs) are disposed in probe 108 in a closely packed and/or alternating fashion, such that (a) a substantial part of each camera's field of view overlaps the field of view of neighboring cameras, and (b) a substantial part of each camera's field of view overlaps the field of illumination of multiple surrounding pattern projectors. Optionally, at least 20%, e.g., at least 50%, e.g., at least 75% of the projected pattern of light are in the field of view of at least one of the cameras at an object focal plane 122 that is located at least 4 mm from the lens that is farthest from the sensor. Due to different possible configurations of the pattern projectors and cameras, some of the projected pattern may never be seen in the field of view of any of the cameras, and some of the projected pattern may be blocked from view by object 112 as the scanner is moved around during a scan.

Rigid structure 106 may be a non-flexible structure to which cameras 104 are coupled so as to provide structural stability to the optics within probe 108. Substrate(s) 150 that includes waveguides may also be coupled to the rigid structure 106 and/or to cameras 104 in embodiments. Coupling all the cameras (and optionally the substrate 150) to a common rigid structure helps maintain geometric integrity of the optics of each pattern projector 102 and each camera 104 under varying ambient conditions, e.g., under mechanical stress as may be induced by the subject's mouth. Additionally, rigid structure 106 helps maintain stable structural integrity and positioning of pattern 102 and cameras 104 with respect to each other.

For some applications, there is at least one uniform light projector 128 (which may be an unstructured light projector that projects non-coherent (e.g., broadband) light across a range of wavelengths) coupled to rigid structure 106. Uniform light projector 128 may transmit white light onto object 112 being scanned. At least one camera, e.g., one of cameras 104. captures two-dimensional color images of object 112 using illumination from uniform light projector 128. Light reflecting off of the object 112 may enter the scanner head and be received by the cameras. The cameras may then generate intraoral scan data based on the received light. In embodiments, one or more near infrared (near-IR) light projectors and/or ultraviolet (UV) unpatterned light projectors may also be included in the probe 108.

Scan data generated by intraoral scanner 100 may be sent (e.g., view a wired or wireless connection) to a processor or processing device 130 of a computing device that may be external to the intraoral scanner 100. The processor 130 may run a surface reconstruction algorithm that may use detected patterns (e.g., dot patterns) projected onto object 112 to generate a 3D surface of the object 112. In some embodiments, the processor 130 may combine at least one 3D scan captured using illumination from structured light projectors 102 with a plurality of intraoral 2D images captured using illumination from uniform light projector 128 in order to generate a digital three-dimensional image of the intraoral three-dimensional surface. Using a combination of structured light and uniform illumination enhances the overall capture of the intraoral scanner and may help reduce the number of options that processor 130 needs to consider when running a correspondence algorithm used to detect depth values for object 112. In one embodiment, the intraoral scanner and correspondence algorithm described in U.S. application Ser. No. 16/446,181, filed Jun. 19, 2019, is used. U.S. application Ser. No. 16/446,181, filed Jun. 19, 2019, is incorporated by reference herein in its entirety.

For some applications, all data points taken at a specific time are used as a rigid point cloud, and multiple such point clouds are captured at a frame rate of over 10 captures per second. The plurality of point clouds are then stitched together using a registration algorithm, e.g., iterative closest point (ICP), to create a dense point cloud. A surface reconstruction algorithm may then be used to generate a representation of the surface of object 112.

For some applications, at least one temperature sensor 132 is coupled to rigid structure 106 and measures a temperature of rigid structure 106. Temperature control circuitry 134 disposed within intraoral scanner 100 (a) receives data from temperature sensor 132 indicative of the temperature of rigid structure 106 and (b) activates a temperature control unit 136 in response to the received data. Temperature control unit 136, e.g., a PID controller, may keep probe 108 at a target temperature (e.g., between 35 and 43 degrees Celsius, between 37 and 41 degrees Celsius, etc.). Keeping probe 108 above 35 degrees Celsius, e.g., above 37 degrees Celsius, reduces fogging of the glass surface of intraoral scanner 100, through which structured light projectors 102 project and cameras 104 view, as probe 108 enters the oral cavity, which is typically around or above 37 degrees Celsius. Keeping probe 108 below 43 degrees, e.g., below 41 degrees Celsius, prevents discomfort or pain.

In some embodiments, heat may be drawn out of the probe 108 via a heat conducting element 140, e.g., a heat pipe, that is disposed within intraoral scanner 100, such that a distal end 145 of heat conducting element 140 is in contact with rigid structure 106 and a proximal end 151 is in contact with a proximal end 160 of intraoral scanner 100. Heat is thereby transferred from rigid structure 106 to proximal end 160 of intraoral scanner 100. Alternatively or additionally, a fan disposed in a handle region of intraoral scanner 100 may be used to draw heat out of probe 108.

In some embodiments, the proximal end 160 of intraoral scanner 100 includes a handle of the intraoral scanner. In some embodiments, the proximal end 160 of intraoral scanner 100 includes at least one of the one or more light sources 154. In an example, the one or more light sources 154 may not be disposed in the probe 108. Alternatively, at least one of the one or more light sources 154 are disposed in the probe 108.

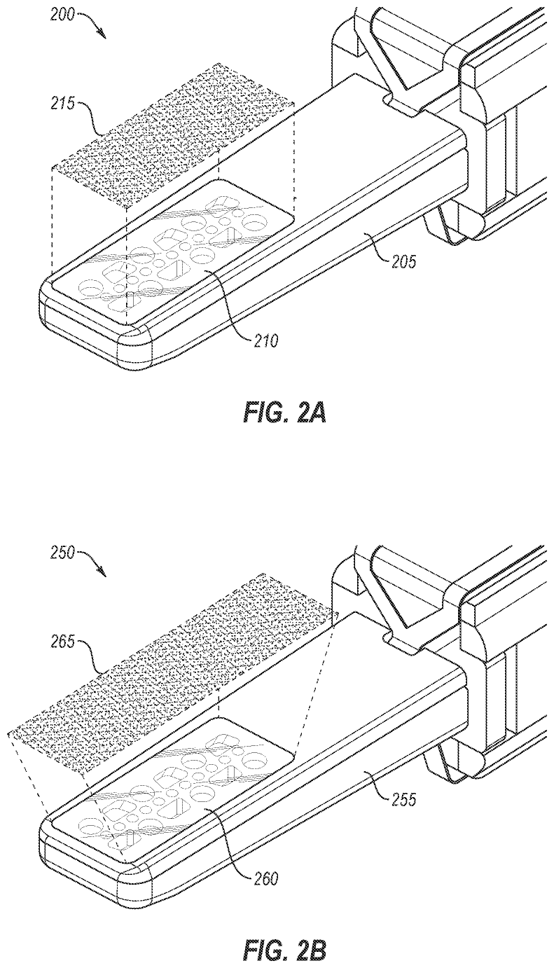

FIG. 2A illustrates a probe 205 of an intraoral scanner 200 and shows a first field of projection 215 of patterned light output by the intraoral scanner 200, in accordance with an embodiment. In embodiments, intraoral 200 corresponds to intraoral scanner 100 of FIG. 1. As shown, the probe 205 includes a window 210 at a distal end of the probe. The window in some embodiments includes one or more waveguides formed therein. The waveguides may include pattern projectors (e.g., FGCs), which may be etched or otherwise formed into a surface of the window 210. The window 210 may be formed from a material that is transparent to light. Examples of materials that may be used for the window 210 include silica, glass, quartz, plastic, polycarbonate, doped glass, silicon, doped silicon, GaAs, AsInP, and so on. Some materials such as glass, doped glass, polycarbonate, quartz and plastic may be transparent to visible light, and may be used for wavelengths of visible light (e.g., blue and green coherent light output by lasers). Some materials such as silicon, doped silicon, GaAs and AsInP may be transparent to wavelengths outside of the visible regime, and may be used, for example, for IR or near-IR radiation. In some embodiments, the window 210 is disposed over one or more waveguides, and thus the waveguides in such embodiments may not be integrated into the window 210. In some embodiments, window 210 includes two windows, where a first window includes waveguides and a second window lacks waveguides. The second window may be placed over the first window, for example, to provide protection, anti-reflection, index of refraction matching to an external environment (e.g., to air), and so on. In order to generate a spot array or other light pattern having the first field of projection 215, the intraoral scanner 200 may include a uniform spread of identical pattern projectors (e.g., FGCs) across the window 210. In one embodiment, the pattern projectors each have a same orientation relative to a longitudinal axis of the probe 205. For example, each of the pattern projectors may be aligned with the longitudinal axis of the probe 205. In one embodiment, the focused light forming the spot array or other pattern has rays that are approximately parallel (e.g., such that a field of projection stays approximately constant over a depth of focus range of the intraoral scanner.

FIG. 2B illustrates a probe 255 of an intraoral scanner 250 and shows a second field of projection 265 of patterned light output by the intraoral scanner 250, in accordance with an embodiment. In embodiments, intraoral scanner 250 corresponds to intraoral scanner 100 of FIG. 1. As shown, the probe 255 includes a window 260 at a distal end of the probe. The window 260 in some embodiments includes one or more waveguides formed therein. The waveguides may include pattern projectors (e.g., FGCs), which may be etched or otherwise formed into a surface of the window 260. The window 260 may be formed from a material that is transparent to light, as set forth above. In some embodiments, the window 260 is disposed over one or more waveguides, and thus the waveguides in such embodiments may not be integrated into the window 260. In order to generate a spot array or other pattern having the second field of projection 265, the intraoral scanner 250 may include a spread of identical pattern projectors (e.g., FGCs) across the window 260. In one embodiment, the pattern projectors have disparate orientations relative to a longitudinal axis of the probe 265. For example, a first subset of the pattern projectors may have a first angle relative to the longitudinal axis, and a second subset of the pattern projectors may have a second angle relative to the longitudinal axis. In one embodiment, the second field of projection 265 is considered a wide field of projection (as compared to the first field or projection 215). The different FGCs may have the same orientation relative to one another but radiate in different angles relative to one another in embodiments. Additionally, or alternatively, the FGCs may have different orientations to one another.

FIG. 3A illustrates a first waveguide configuration 300 for use in an intraoral scanner, in accordance with an embodiment. In one embodiment, the first waveguide configuration 300 is used in intraoral scanner 200 of FIG. 2A. For example, the first waveguide configuration 300 may be integrated into window 210 (or a region of window 210) of FIG. 2A. In embodiments, first waveguide configuration 300 produces first field of projection 215.

First waveguide configuration 300 includes one or more first waveguides 308A and one or more second waveguides 308B integrated into a transparent substrate (e.g., fused silica) 312. In one embodiment, the transparent substrate has a width of about 2 mm. Other widths are also easily achievable. Each of the waveguides 308A-B include a transport region 310A-B, a taper region 315A-B, and a projection region (pattern projector) 320A-B. The transport region 310A-B may be substantially longer and thinner than the other regions. The projection region 320A-B may have a width and length configured based on a pattern to be projected by the projection region 320A-B and/or on a target depth of field. The projection region 320A-B may be significantly wider and shorter than the transport region 310A-B. The taper region 315A-B may transition a width of the waveguides 308A-B between a first width of the transport region 310A-B and a second width of the projection region 320A-B. In one embodiment, the FGC is configured to receive blue and/or green light (e.g., light having a wavelength of about 520 nm and/or light having a wavelength of about 450 nm). For light having a wavelength of 520 nm, the FGC may have grooves with a period of about 315 nm in embodiments. For light having a wavelength of about 450 nm, the FGC may have grooves with a period of about 270 nm in embodiments. The length of the FGCs may be based at least in part on depth of field considerations, and may be, for example, hundreds of microns (e.g., about 150 microns). In one embodiment, the transport region 310A-B has a width and depth of about 3-5 microns, and the FGC has grooves with a depth of about 100 nm.

As shown, waveguides 308A may be configured to receive and propagate first coherent light 305A having a first wavelength, and waveguides 308B may be configured to receive and propagate second coherent light 305B having a second wavelength. Additionally, two or more wavelengths of light may be propagated through the same waveguide. Additionally, or alternatively, a single source with a wide spectrum of radiation may be coupled to the waveguide and propagate a spectrum of wavelengths through the waveguide. In some embodiments, a separate light source inputs light into each of the waveguides 310A and a single light source inputs light into a single waveguide 310B, which splits the light into multiple (e.g., three) sub-waveguides. Each light source may optically connect to a waveguide having a single pattern projector 320A-B or to a waveguide that splits into multiple sub-waveguides and that includes multiple pattern projectors 320A-B. In first waveguide configuration 300, the waveguides 308 A-B are approximately aligned with a longitudinal axis of the substrate 312 (and of a probe that includes the substrate 312), which may correspond to a Y axis. Furthermore, each of the projection regions 320A-B (e.g., FGCs) share a same orientation that is aligned with the longitudinal axis of the substrate 312. In some embodiments, the transport region of a waveguide curve in one or more directions (e.g., are not necessarily straight lines).

In embodiments, first waveguide configuration 300 only shows a fraction of the number of waveguides and corresponding pattern projectors 320A-B (e.g., FGCs) of those waveguides included in the intraoral 200 (e.g., in window 210). Moreover, the relative positioning (e.g., spacing, position in substrate 312, etc.) of the waveguides 308A-B and their projection regions 320A-B may vary.

FIG. 3B illustrates a second waveguide configuration for use in an intraoral scanner, in accordance with an embodiment. In one embodiment, the second waveguide configuration 350 is used in intraoral scanner 250 of FIG. 2B. For example, the second waveguide configuration 300 may be integrated into window 260 of FIG. 2B. In embodiments, second waveguide configuration 350 produces second field of projection 265.

Second waveguide configuration 350 includes one or more first waveguides 368A and one or more second waveguides 368B integrated into a transparent substrate (e.g., fused silica) 362. In one embodiment, the transparent substrate has a width of about 2 mm. Other widths are also easily achievable. Each of the waveguides 368A-B include a transport region 360A-B, a taper region 365A-B, and a projection region (pattern projector) 370A-B. The transport region 360A-B may be substantially longer and thinner than the other regions, and may be curved in embodiments. The projection region 370A-B may have a width and length configured based on a pattern and/or depth of field to be projected by the projection region 370A-B. The projection region 370A-B may be significantly wider and shorter than the transport region 360A-B. The taper region 365A-B may transition a width of the waveguides 368A-B between a first width of the transport region 360A-B and a second width of the projection region 370A-B.

As shown, waveguides 368A may be configured to receive and propagate first coherent light 355A having a first wavelength, and waveguides 368B may be configured to receive and propagate second coherent light 355B having a second wavelength. In some embodiments, a separate light source inputs light into each of the waveguides 360A-B. In some embodiments, a single light source inputs light into a single waveguide 360A-B, which splits the light into multiple sub-waveguides. In some embodiments, multiple light sources are coupled to the same waveguide and project different wavelengths of radiation into the waveguide (e.g., either sequentially or in parallel). Each light source may optically connect to a waveguide having a single pattern projector 370A-B or to a waveguide that splits into multiple sub-waveguides and that includes multiple pattern projectors 370A-B. In second waveguide configuration 350, the waveguides 368A-B are angled relative to a longitudinal axis of the substrate 362 (and of a probe that includes the substrate 362), which may correspond to a Y axis. Furthermore, the projection regions 370A-B (e.g., FGCs) may be divided into two or more groups, wherein projection regions in a group 370A-B share a same orientation that is angled relative to the longitudinal axis of the substrate 362.

In embodiments, second waveguide configuration 350 only shows a fraction of the number of waveguides and corresponding pattern projectors 370A-B (e.g., FGCs) of those waveguides included in the intraoral scanner 250 (e.g., in window 260). Moreover, the relative positioning (e.g., spacing, position in substrate 362, etc.) of the waveguides 368A-B and their projection regions 370A-B may vary.

FIG. 3C illustrates a third waveguide configuration 380 for use in an intraoral scanner, in accordance with an embodiment. In one embodiment, the third waveguide configuration 380 is used in intraoral scanner 200 of FIG. 2A or intraoral scanner 250 of FIG. 2B. For example, the third waveguide configuration 380 may be integrated into window 210 of FIG. 2A or window 260 of FIG. 2B.

Third waveguide configuration 380 includes at least one waveguide 398 integrated into a transparent substrate (e.g., fused silica) 382. Waveguide 398 includes multiple transport regions, including a first transport region 390A that receives first light 385A having a first wavelength from a first light source and a second transport region 390B that receives second light 385B having a second wavelength from a second light source 385B. The first and second transport regions 390A-B may merge into a single transport region that is connected to a first taper region 391A that connects the transport region to an input end of a projection region 394A. Alternatively, a single transport region may couple to two different light sources without the use of two transport regions that merge. An output end of the projection region 394A may connect to a second taper region 391B that tapers from a width of the projection region 394A down to a width of another transport region 390C. Another taper region 291A may then widen out from the width of the transport region 390C to the width of a next projection region 394A. This sequence may continue multiple times to form two, three, four, five, six, seven or more projection regions 394A-B in series. In some embodiments, the first wavelength of light may only propagate through a first subset of the projection regions 392A (including projection regions 394A), and the second wavelength of light may pass through the first subset of projection regions 392A and also propagate through a second subset of projection regions 392B (including projection regions 394B).

In one embodiment, as shown, each of the projection regions 394A is aligned with a longitudinal axis of the substrate 382 (and of a probe in which the substrate is mounted). Alternatively, the projection regions 394A may have different orientations relative to the longitudinal axis.

In embodiments, third waveguide configuration 380 only shows a fraction of the number of waveguides and corresponding pattern projectors 3904A-B (e.g., FGCs) of those waveguides included in the intraoral scanner. Moreover, the relative positioning (e.g., spacing, position in substrate 382, etc.) of the waveguide(s) 398 and their projection regions 394A-B may vary. The length and orientation (e.g., curves, angles of lines, etc.) of each of the transport regions 390C may vary.

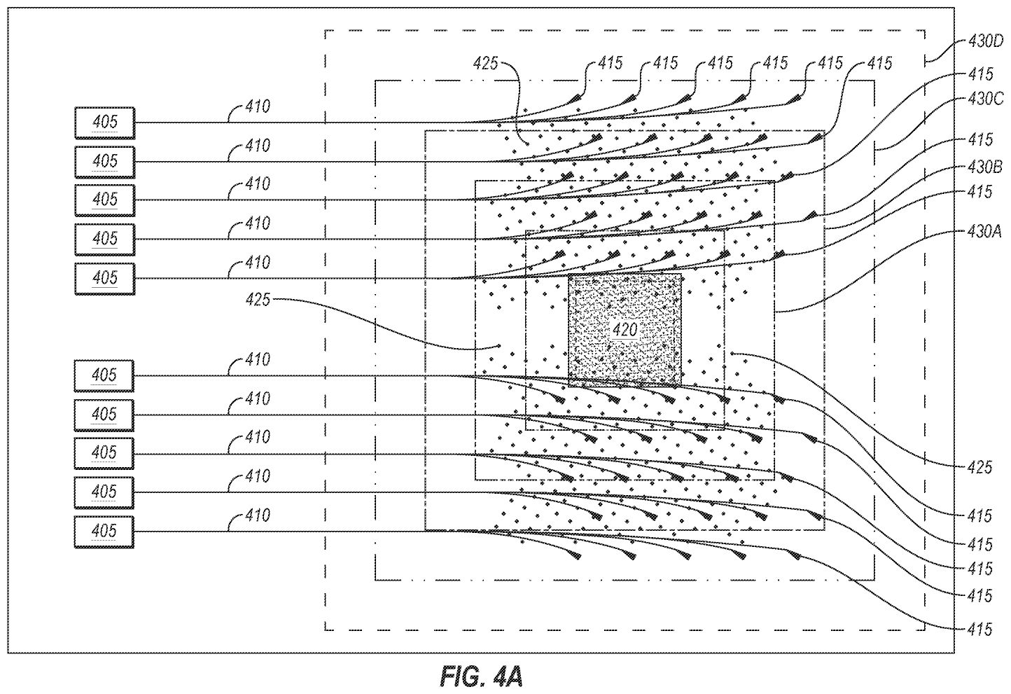

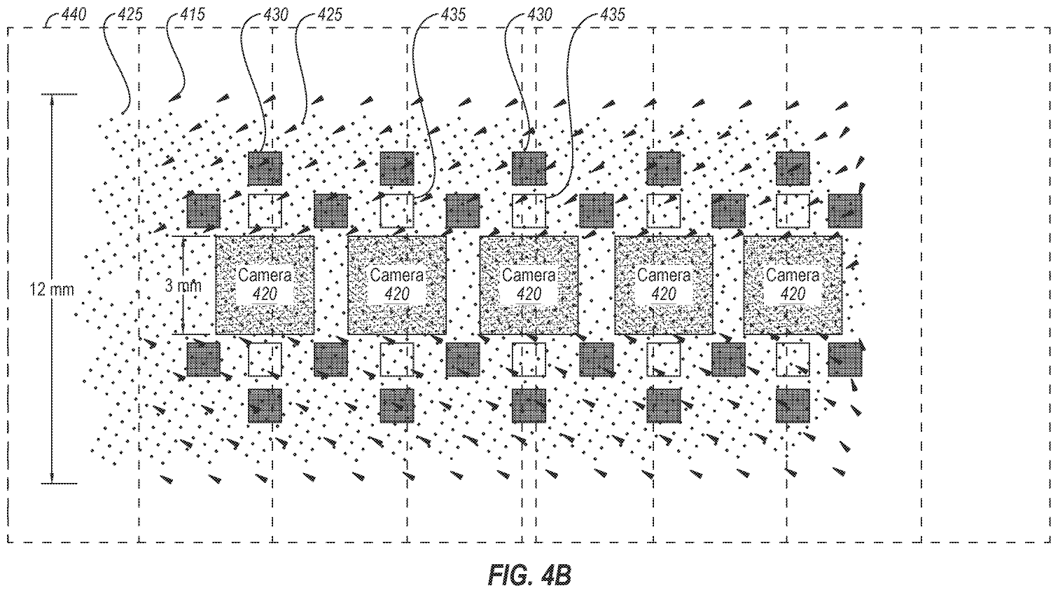

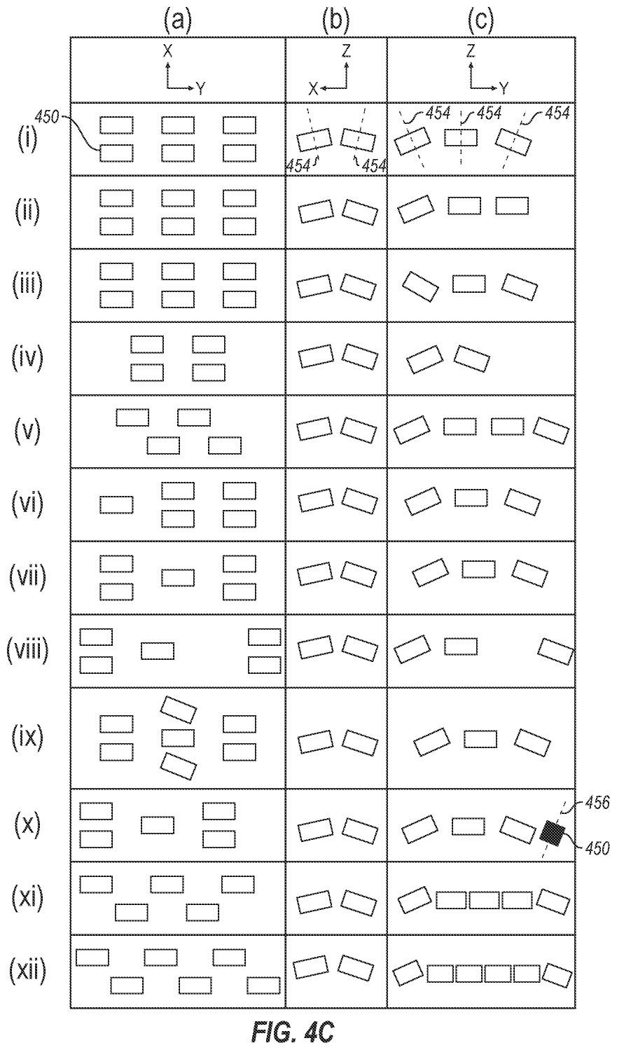

FIG. 4A illustrates an example layout of a camera 420, surrounding waveguides 410 and one or more light sources 405 in an intraoral scanner, in accordance with an embodiment. As shown, waveguides 410 receive coherent light having a preconfigured wavelength from one or more light sources 405 (e.g., one or more semiconductor lasers). As shown, each waveguide 410 receives light from a different light source 405. However, some or all of the waveguides 410 may receive light from the same light source 405. Each of the waveguides includes multiple projection regions 415 (e.g., multiple FGCs) laid out in a substrate. The arrangement of projection regions 415 causes a light pattern (e.g., an array of spots 425) to be projected into the field of view of the camera 420. As shown, none of the waveguides 410 or their projection regions 415 overlay the camera, providing the camera with an unobstructed field of view. The waveguides 410 and projection regions 415 may be arrayed around the camera to provide a projected light pattern (e.g., array of spots) to be imaged by the camera 420. A lateral spacing between cameras and pattern projectors may be less than a lateral spacing between cameras and traditional structured light projectors due to the size of such traditional structured light projectors. A minimum lateral distance (e.g., a distance in the X-Y plane) between camera 420 and a closest projection region 415 is about 1-2 mm in embodiments, or less than 2 mm in embodiments, or less than 1 mm in embodiments.

Concentric squares show fields of view 430A-D of the camera 420 at different distances from the camera 420. In one embodiment, the camera 420 has a field of view 430A at a distance of 6 mm, a field of view 430B at a distance of 8 mm, a field of view 430C at a distance of 10 mm, and a field of view 430D at a distance of 12 mm.

FIG. 4A illustrates an arrangement having only a single camera and a surrounding fifty projection regions 415. However, in embodiments the intraoral scanner would include multiple cameras. These cameras may all be arranged in a single row or on multiple rows. The cameras may have a same orientation or may have different orientations.

FIG. 4B illustrates an example layout of a set of cameras 420 in a single line, in accordance with an embodiment. FIG. 4C shows multiple alternate example layouts of a set of cameras.

Referring to FIG. 4B, cameras 420 are arranged in a single line or row along a longitudinal axis of a probe for an intraoral scanner. Arranged about the cameras are projection regions 415 (e.g., FGCs) of waveguides, each of which outputs patterned light (e.g., an array of spots). In the aggregate, the patterns output by each of the projection regions 415 form a larger projection region that spans the fields of view of each of the cameras 420. In the example layout, the cameras have a footprint of about 3 mm×3 mm, and a field of projection of the output patterned light has a width of about 12 mm. In the illustrated example, the intraoral scanner includes 160 projection regions arranged about the cameras 420, which together generate an array of 1440 spots. In the example, the cameras have a field of view 440 at a distance of 12 mm from a camera lens plane. The intraoral scanner may additionally include multiple non-coherent light sources 430 (e.g., white light sources) arrayed about the cameras 420 and/or multiple near-IR or IR light sources 435 arrayed about the cameras 420. Accordingly, the cameras may generate images of the pattern of light projected onto an imaged object, the imaged object illuminated by IR or near-IR radiation, and color images of the imaged object (while the object is illuminated by white light).

Reference is now made to FIG. 4C, which is a chart depicting a plurality of different configurations for the position of cameras 450 in a probe of an intraoral scanner, in accordance with some embodiments of the present disclosure. Cameras 450 are represented in FIG. 4C by rectangles. It is noted that rectangles are used to represent the cameras, since typically, each camera sensor and the field of view β (beta) of each camera 450 have aspect ratios of 1:2. Column (a) of FIG. 4C shows a bird's eye view of the various configurations of cameras 450. The Y-axis as labeled in the first row of column (a) corresponds to a central longitudinal axis of the intraoral scanner's probe. Column (b) shows a side view of cameras 450 from the various configurations as viewed from a line of sight that is coaxial with the central longitudinal axis of the probe. Column (b) shows cameras 450 positioned so as to have optical axes 454 at an angle of 90 degrees or less, e.g., 35 degrees or less, with respect to each other. Column (c) shows a side view of cameras 450 of the various configurations as viewed from a line of sight that is perpendicular to the central longitudinal axis of the probe.

Typically, the distal-most (toward the positive Y-direction in FIG. 4C) and proximal-most (toward the negative Y-direction in FIG. 4C) cameras 450 are positioned such that their optical axes 454 are slightly turned inwards, e.g., at an angle of 90 degrees or less, e.g., 35 degrees or less, with respect to the next closest camera 450. The camera(s) 450 that are more centrally positioned, i.e., not the distal-most camera 450 nor proximal-most camera 450, are positioned so as to face directly out of the probe, their optical axes 454 being substantially perpendicular to the central longitudinal axis of the probe.

Typically, the number of cameras 450 in the probe may range from four, e.g., as shown in rows (iv) and (v), to seven, e.g., as shown in row (ix). It is noted that the various configurations shown in FIG. 4C are by way of example and not limitation, and that the scope of the present disclosure includes additional configurations not shown. For example, the scope of the present disclosure includes more than seven cameras positioned in the probe.

In an example application, an apparatus for intraoral scanning (e.g., an intraoral scanner) includes an elongate handheld wand comprising a probe at a distal end of the elongate handheld wand, multiple waveguides that include an array of FGCs disposed within the probe, and at least four cameras disposed within the probe. Each FGC may generate a pattern of light when the light is transmitted through the FGC. Each of the at least four cameras may include a camera sensor and one or more lenses, wherein each of the at least four cameras is configured to capture a plurality of images that depict at least a portion of the projected pattern of light on an intraoral surface. In one embodiment, the at least four cameras may be arranged in at least two rows that are each approximately parallel to a longitudinal axis of the probe, the at least two rows comprising at least a first row and a second row.

In a further application, a distal-most camera along the longitudinal axis and a proximal-most camera along the longitudinal axis of the at least four cameras are positioned such that their optical axes are at an angle of 90 degrees or less with respect to each other from a line of sight that is perpendicular to the longitudinal axis. Cameras in the first row and cameras in the second row may be positioned such that optical axes of the cameras in the first row are at an angle of 90 degrees or less with respect to optical axes of the cameras in the second row from a line of sight that is coaxial with the longitudinal axis of the probe. A remainder of the at least four cameras other than the distal-most camera and the proximal-most camera have optical axes that are substantially parallel to the longitudinal axis of the probe. Each of the at least two rows may include an alternating sequence of light projectors and cameras.

In a further application, the distal-most camera along the longitudinal axis and the proximal-most camera along the longitudinal axis are positioned such that their optical axes are at an angle of 35 degrees or less with respect to each other from the line of sight that is perpendicular to the longitudinal axis. The cameras in the first row and the cameras in the second row may be positioned such that the optical axes of the cameras in the first row are at an angle of 35 degrees or less with respect to the optical axes of the cameras in the second row from the line of sight that is coaxial with the longitudinal axis of the probe.

In a further application, the at least four cameras may have a combined field of view of 25-45 mm along the longitudinal axis and a field of view of 20-40 mm along a z-axis corresponding to distance from the probe.

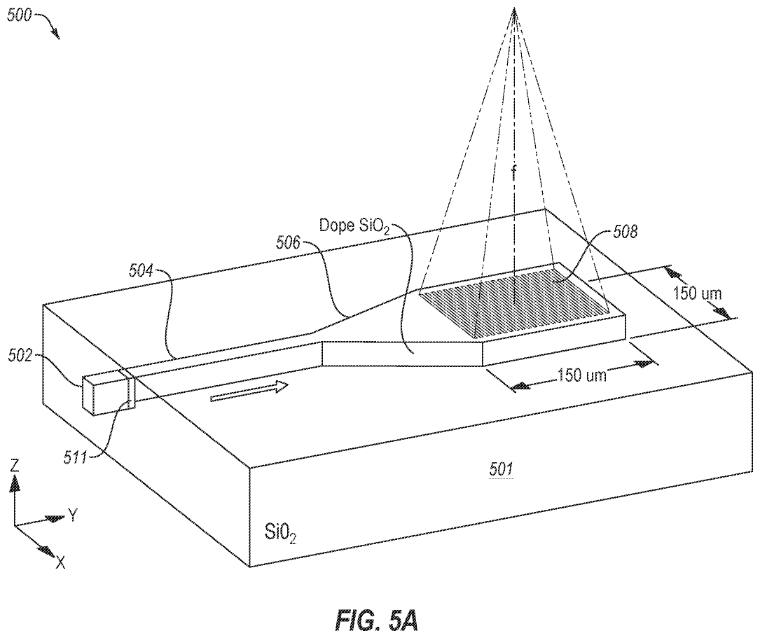

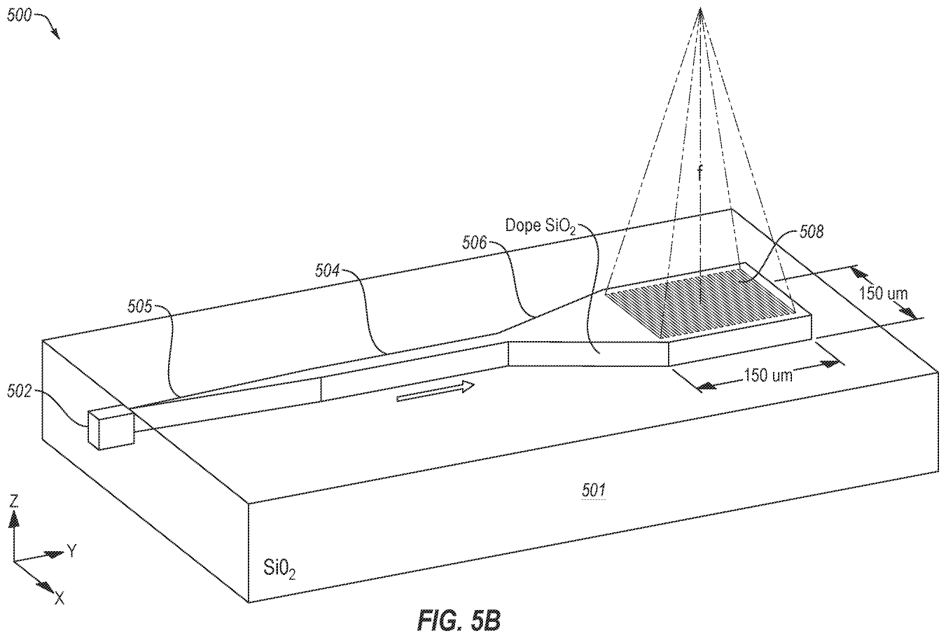

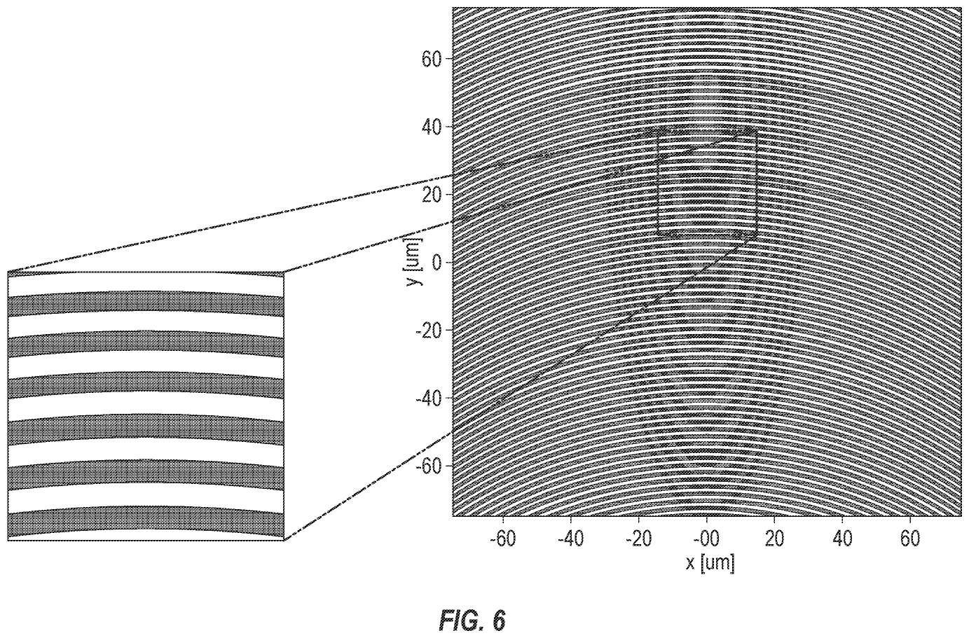

FIG. 5A illustrates a perspective view of an example waveguide 500 that includes a focusing grating coupler 508 coupled to a laser 502 via a coupling 511, in accordance with an embodiment. The waveguide 500 is formed in a substrate 501 (e.g., an SiO₂ or glass substrate). A region of the substrate 501 may be doped to form the waveguide 500. A laser 502 may be coupled to the waveguide 500, and may project light into the waveguide. In some embodiments, the laser 502 may be coupled to the transport region 504 of the waveguide 500 via a coupling 511, which may include at least one of a lens, a prism or a grating.

The waveguide 500 includes a transport region 504, followed by a taper region 506, and ending in an FGC 508. In the illustrated example, the FGC 508 has a width and length of 150 microns, and the transport region 504 of the waveguide has a width and depth of about 1-5 microns. However, other FGC sizes and/or transport region sizes may also be used. The FGC 508 may include a pattern of gratings formed in a surface of the substrate 501 that focus a pattern of light at a focal depth f. The FGC 508 may be designed such that the focal length f is a focal range that corresponds to a focal range of a camera. In embodiments, patterned light output by the FGC 508 has an approximately constant overlap (and thus an approximately constant focus) over an entire depth of focus of the cameras of the intraoral scanner.

The waveguide 500 may guide optical waves with low loss to surrounding media. The doped area of the substrate 501 forming the waveguide may have a depth with a sub-micron size in embodiments. The waveguide 500 can be designed for a specific wavelength or mode. A first mode distribution may be similar to a Gaussian beam distribution. The doped region of the substrate may be, for example, ion-doped with one or more conventional dopants, and may be formed via nano-fabrication processes.

FIG. 5B illustrates a perspective view of an example waveguide 500 that includes a focusing grating coupler 508 coupled to a laser 502 without use of a coupling, in accordance with an embodiment. The waveguide 500 is formed in a substrate 501 (e.g., an SiO₂ or glass substrate). A region of the substrate 501 may be doped to form the waveguide 500 and/or may be etched to form the waveguide 500. A laser 502 may be coupled to the waveguide 500, and may project light into the waveguide. In one embodiment, light source(s) 154 optically couple to one or more waveguides directly (e.g., by direct connection between a laser die or optical fiber and an edge of a waveguide).

The waveguide 500 further includes a taper region 505 and transport region 504, followed by an additional taper region 506, and ending in an FGC 508. The taper region 505 may taper to a narrow width (e.g., to a point) in embodiments. In some embodiments, multiple tapered edge couplers (e.g., multiple tapered regions 505) such as 3-5 tapered edge couplers (e.g., 3-5 tapered regions) may be fabricated to merge into a single transport region 504. This may increase a coupling efficiency of the waveguide. The multiple tapered regions 505 may each receive light from the laser 502 and may merge the light at transport region 504 in embodiments. In the illustrated example, the FGC 508 has a width and length of 150 microns, and the taper region 505 of the waveguide has a width and depth of about 1-5 microns where it meets the transport region. Transport region may have a width and depth of about 1-5 microns in embodiments. However, other FGC sizes and/or transport region sizes and/or taper region sizes may also be used. The FGC 508 may include a pattern of gratings formed in a surface of the substrate 501 that focus a pattern of light at a focal depth f. The FGC 508 may be designed such that the focal length f is a focal range that corresponds to a focal range of a camera. In embodiments, patterned light output by the FGC 508 has an approximately constant overlap (and thus an approximately constant focus) over an entire depth of focus of the cameras of the intraoral scanner.

The waveguide 500 may guide optical waves with low loss to surrounding media. The doped area of the substrate 501 forming the waveguide may have a depth with a sub-micron size in embodiments. The waveguide 500 can be designed for a specific wavelength or mode. A first mode distribution may be similar to a Gaussian beam distribution. The doped region of the substrate may be, for example, ion-doped with one or more conventional dopants, and may be formed via nano-fabrication processes.

FIG. 6 illustrates a top view of a focusing grating coupler for use in a waveguide, in accordance with an embodiment. As shown, the FGC includes a pattern of grooves etched within the substrate. The grooves may be curved and/or chirped such that their average period is on the scale of the wavelength of light that will be shone through the waveguide 500. The depth of the grooves may be on the order of a hundred to a few hundred nanometers in embodiments. This configuration causes a propagating mode of projected light traveling through the waveguide 500 to be emitted at an angle to a plane of the substrate 500 (e.g., approximately orthogonal to the plane) and into free space, and to focus at the focal distance fin the Z-direction (orthogonal to the plane of the substrate 500), in some embodiments according to holographic principles. In general, the FGC forces light to couple out of the waveguide. A period and curvature of the grooves may be designed for a target wavelength of light, a target projected pattern, a target focal distance (or range of focal distances), and output angle (e.g., angle relative to the plane of the substrate 508) and/or relative to the Y-axis). To generate a pattern of one or more projected spots, the pattern in the FGC may resemble concentric ovals or curves in embodiments as shown. In the illustration, dark areas represent the surface of the substrate, and light areas are the grooves etched into the substrate. The left-hand rectangle shows a zoomed-in view of a small region within the focusing grating coupler.

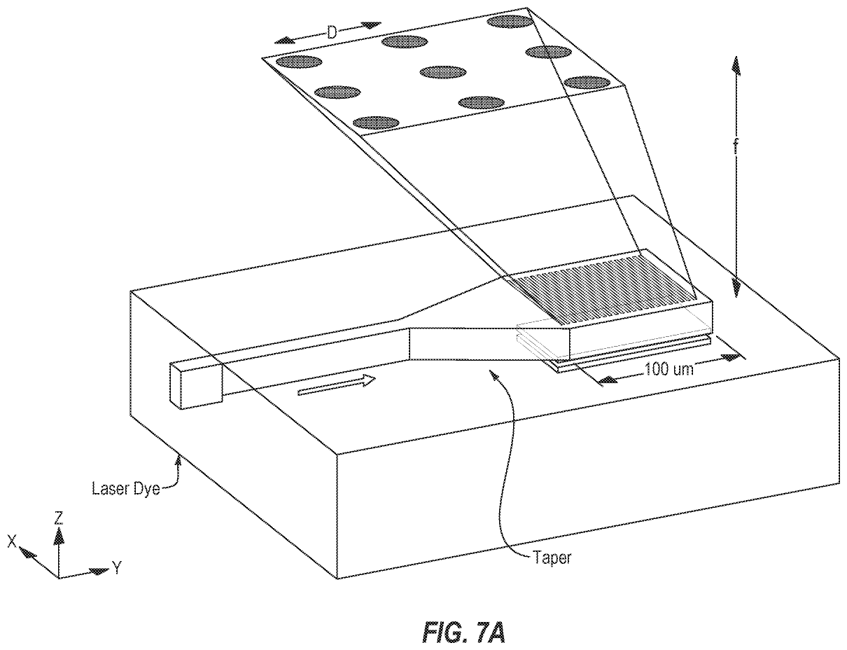

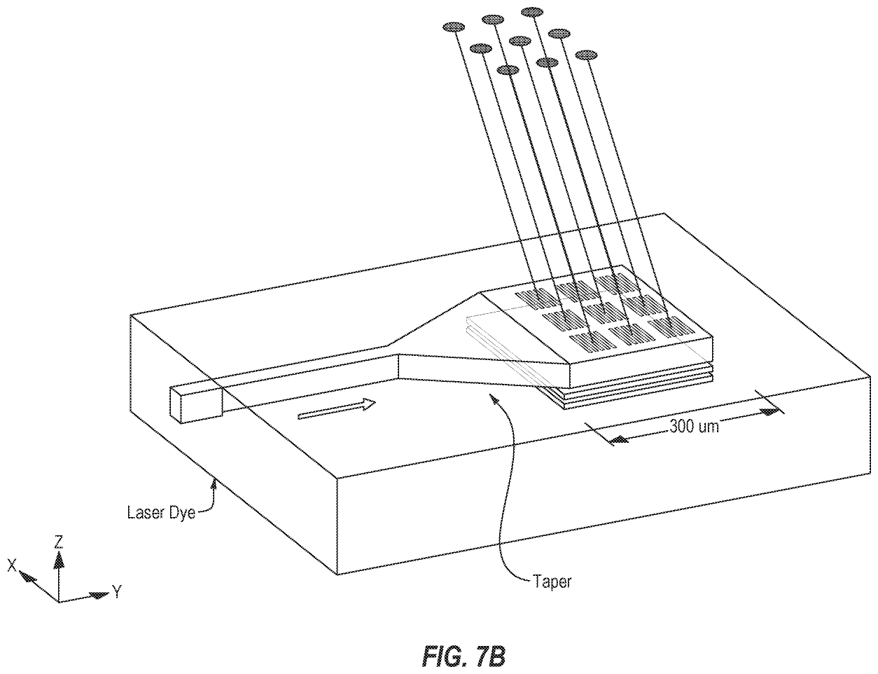

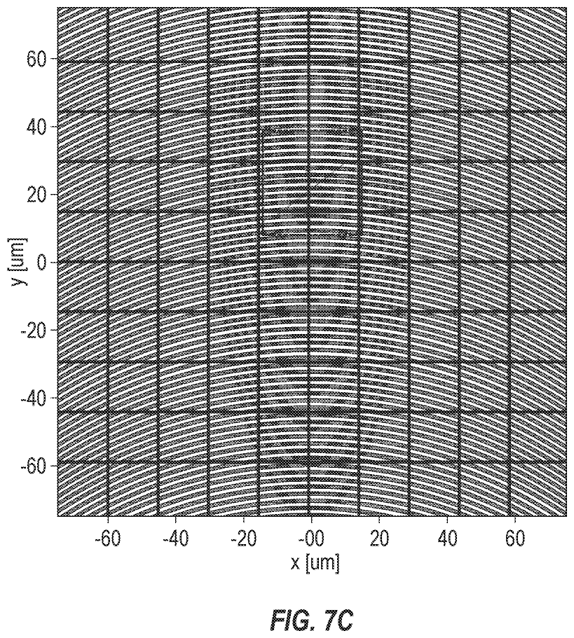

FIG. 7A illustrates a perspective view of an example waveguide that includes a focusing grating coupler configured to output patterned light having a first focused spot pattern, in accordance with an embodiment. The FGC may be divided into a grid of regions (e.g. of squares or rectangles). In each region of the grid, the grooves of the FGC may be dislocated by, for example, about 40-100 nm along the Y-axis with respect to neighboring regions. This modification of the FGC pattern causes out-coupled light (e.g., an out-coupled beam) to split into multiple focused beams (e.g., 9 focused beams) with the same focal distance as the out-coupled beam of an unperturbed FGC. In one embodiment, an efficiency of an FGC that divides the out-coupled mode into 9 spots is about 31%, with an efficiency of about 3.5% for each individual spot.

FIG. 7C illustrates an FGC region divided into a grid of 10×10 squares, which produces an array of 9 focused spots arranged in a 3×3 array. In an embodiment, curved grating areas that give different phases to outcoupled light are achieved by shifting the grating patterns by a fixed amount (e.g., 40-80 nm, about 60 nm, etc.) between grating patterns of adjacent areas in the grid. FIG. 7A shows the 3×3 array of focused spots. The focused spots are evenly spaced, having a spacing D between adjacent spots at a focal distance f.

FIG. 7B illustrates a perspective view of an example waveguide that includes a set of focusing grating couplers (e.g., nine FGCs each similar to the FGC in FIG. 5) configured to output patterned light having a second focused spot pattern, in accordance with an embodiment. In one embodiment, the FGC is shown with a reflector disposed below the FGC to increase outcoupling efficiency. In an embodiment, the FGC has a size of about 100×100 microns. In one embodiment, the FGC has a length of about 300 microns. In one embodiment, the FGC has an efficiency of about 50%.

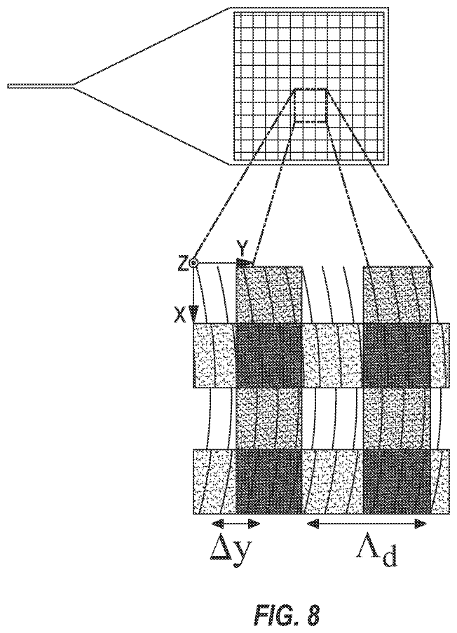

FIG. 8 illustrates a top view of a focusing grating coupler design that outputs a spot pattern, in accordance with an embodiment. The FGC design shown has a length and width of about 100 microns, and is divided into patches having a periodicity Ad of about 10 microns and a grating offset between adjacent patches Ay of about 85 nm. The illustrated FGC has an efficiency of about 30% for a projected pattern of 9 spots. To produce, for example, 600 spots, 8×8 FGCs may be used, having a total size of about 0.8×0.8 mm.

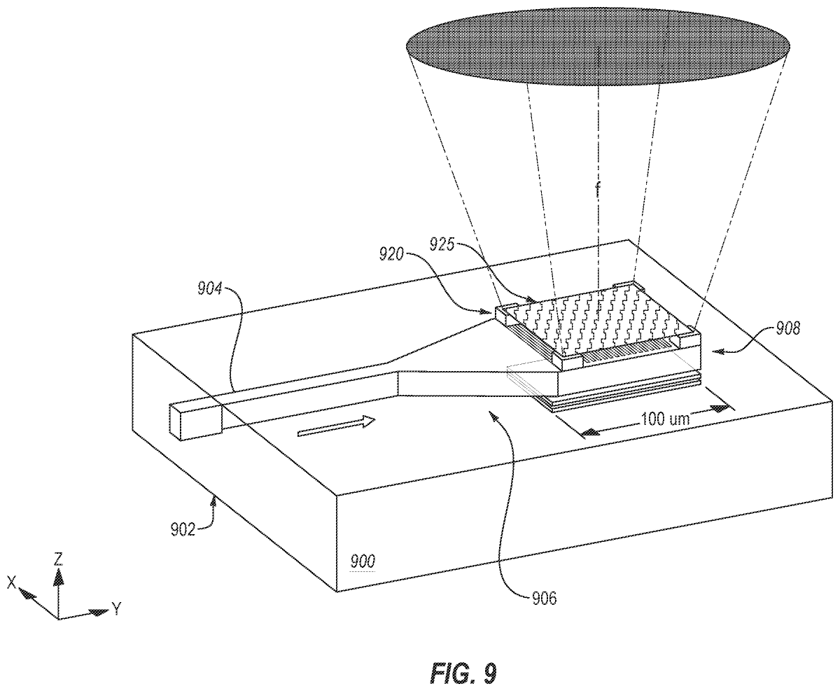

FIG. 9 illustrates a perspective view of an example waveguide that includes a focusing grating coupler 908 configured to output patterned light having a second focused spot pattern and/or other pattern and a microlens array 925 over the focusing grating coupler 908, in accordance with an embodiment. As shown, a laser 902 may be coupled to the waveguide. The waveguide may include a transport region 904, a taper region 906 and the FGC 908. One or more spacers 920 (optionally formed from fused silica, glass, or other materials) may position and hold the microlens array 925 over the FGC 908. The microlens array 925 may divide an out-coupled beam emerging from the FGC into several modes with different angles, according to a period and refractive index of the microlens array (MLA), according to the diffraction grating principle. Accordingly, a number of spots or other patterns may be increased using the addition of microlens arrays over the FGCs in embodiments. In one embodiment, a single FGC used with an MLA has a length of about 100 microns and an efficiency of about 50%.

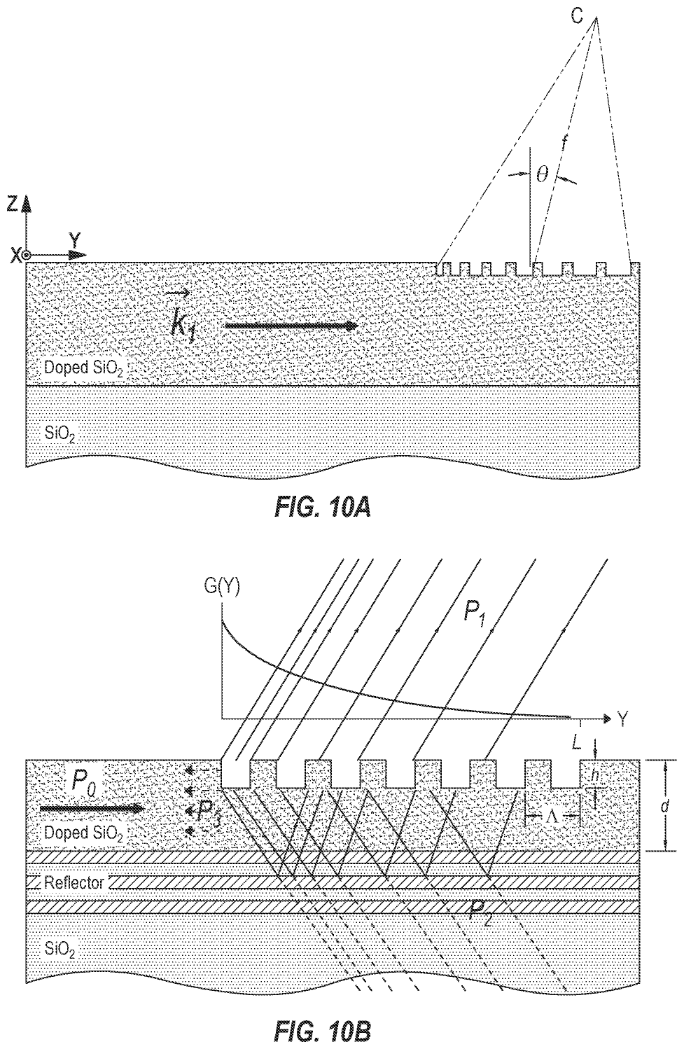

FIGS. 10A-B illustrate views of a focus grating coupler that show how to design a focus grating coupler for use in an intraoral scanner, in accordance with an embodiment. Referring to FIG. 10A, varying a grating period allows different angles θ of out-coupled light such that the FGC acts as a simple lens. The FGC acts as a lens, with F#=f/D, where D is the size of the FGC, and f is the focal length. A focal length of 1.5 mm up to several cm can be achieved using an FGC, with a beam focus reaching a diffraction limit and/or a beam diameter as low as 1 micron. Coupling efficiency of up to 90% has been demonstrated. The grating pattern of the FGC is similar to a holographic recording pattern. The pattern is an interference pattern of a beam of a desired pattern (e.g., spots, lines, etc.) with a guided wave. For a focused beam (spherical wave), the grating pattern resembles a Fresnel zone plate pattern for the specific wavelength. Turning to the figure, k₁ is the incident guided wave vector, c is the focused wave, f is the focal length, and θ is the out-coupled angle. In the illustrated example, the waveguide is formed of doped SiO₂ on an SiO₂ substrate. However, the waveguide and substrate may be any of the materials mentioned herein.

Turning to FIG. 10B, coupling efficiency of an out-coupled mode power P₁ is reduced by two parasitic modes having powers P₂ and P₃, where P₂ is a power of the substrate-coupled mode lost through a bottom of the FGC and P₃ is the power of a back-scattered mode lost by back reflection in the longitudinal direction of the FGC. The amount of power of the pattern or other light coupled out of the FGC, and thus the efficiency for the FGC, can be calculated according to the following equation:

$$η = \frac{{α}_{1}}{{α}_{t o t}} ( 1 - {e}^{- 2 {α}_{t o t} L} )$$

Where α₁ is a coupling coefficient to the outcoupled mode αₜₒₜ is a total of coupling coefficients for all modes, and L is lateral position (i.e. grating length) along the longitudinal axis (Y-axis) into the FGC. As shown, G(y) is an intensity distribution of P₁, showing that for a grating having equal depths h for each trough, an unchanging duty cycle dc and an unchanging grating period Λ, P₁ decreases with increasing length Y into the FGC. Reduction of parasitic modes P₂ and P₃ increases an intensity of out-coupled mode P₁. The parasitic mode P₂ can be reduced by adding a reflecting substrate or coating (e.g., a metallic layer of Bragg reflector composed of several layers of alternating reflectance) that will reflect P₂ towards the grating. In embodiments, the reflector is constructed such that P₂ interferes constructively with P₁. Even without a reflector, but with a waveguide depth permits constructive interference, the efficiency may increase.

Increasing the depth of a particular trench of the FGC increases the intensity of light emitted by that portion of the grating. Accordingly, an intensity uniformity of the FGC can be increased by gradually adjusting the depth of trenches, such that the depth increases with length L. This can cause the intensity distribution G(y) to become an approximately horizontal line. Similarly, the duty cycle dc also affects efficiency. In one embodiment, both the depth of trenches and the duty cycle dc can be adjusted over the length L of the FGC to provide an even intensity distribution g(y).

As discussed earlier, changing the wavelength of light shone into an FGC causes the focal length, angle of the out-coupled mode, and output pattern to change. In embodiments, two or more different wavelengths of light may be shone through the FGCs serially or in parallel to generate different projected light patterns.

According to an example, a user (e.g., a practitioner) may subject a patient to intraoral scanning. In doing so, the user may apply intraoral scanner 100 to one or more patient intraoral locations. The scanning may be divided into one or more segments. As an example, the segments may include a lower dental arch of the patient, an upper dental arch of the patient, one or more preparation teeth of the patient (e.g., teeth of the patient to which a dental device such as a crown or other dental prosthetic will be applied), one or more teeth which are contacts of preparation teeth (e.g., teeth not themselves subject to a dental device but which are located next to one or more such teeth or which interface with one or more such teeth upon mouth closure), and/or patient bite (e.g., scanning performed with closure of the patient's mouth with the scan being directed towards an interface area of the patient's upper and lower teeth). Via such scanner application, the scanner 100 may provide intraoral scan data to a computing device. The intraoral scan data may be provided in the form of intraoral scan/image data sets, each of which may include 2D intraoral scans/images and/or 3D intraoral scans/images of particular teeth and/or regions of an intraoral site. In one embodiment, separate scan/image data sets are created for the maxillary arch, for the mandibular arch, for a patient bite, and for each preparation tooth. Alternatively, a single large intraoral scan/image data set is generated (e.g., for a mandibular and/or maxillary arch). Such scans/images may be provided from the scanner to the computing device in the form of one or more points (e.g., one or more pixels and/or groups of pixels). For instance, the scanner 100 may provide such a 3D scan/image as one or more point clouds.

The manner in which the oral cavity of a patient is to be scanned may depend on the procedure to be applied thereto. For example, if an upper or lower denture is to be created, then a full scan of the mandibular or maxillary edentulous arches may be performed. In contrast, if a bridge is to be created, then just a portion of a total arch may be scanned which includes an edentulous region, the neighboring preparation teeth (e.g., abutment teeth) and the opposing arch and dentition. Additionally, the manner in which the oral cavity is to be scanned may depend on a doctor's scanning preferences and/or patient conditions.

By way of non-limiting example, dental procedures may be broadly divided into prosthodontic (restorative) and orthodontic procedures, and then further subdivided into specific forms of these procedures. Additionally, dental procedures may include identification and treatment of gum disease, sleep apnea, and intraoral conditions. The term prosthodontic procedure refers, inter alia, to any procedure involving the oral cavity and directed to the design, manufacture or installation of a dental prosthesis at a dental site within the oral cavity (intraoral site), or a real or virtual model thereof, or directed to the design and preparation of the intraoral site to receive such a prosthesis. A prosthesis may include any restoration such as crowns, veneers, inlays, onlays, implants and bridges, for example, and any other artificial partial or complete denture. The term orthodontic procedure refers, inter alia, to any procedure involving the oral cavity and directed to the design, manufacture or installation of orthodontic elements at a intraoral site within the oral cavity, or a real or virtual model thereof, or directed to the design and preparation of the intraoral site to receive such orthodontic elements. These elements may be appliances including but not limited to brackets and wires, retainers, clear aligners, or functional appliances.

During an intraoral scan session, an intraoral scan application receives and processes intraoral scan data (e.g., intraoral scans) and generates a 3D surface of a scanned region of an oral cavity (e.g., of a dental site) based on such processing. To generate the 3D surface, the intraoral scan application may register and “stitch” or merge together the intraoral scans generated from the intraoral scan session in real time or near-real time as the scanning is performed. In one embodiment, performing registration includes capturing 3D data of various points of a surface in multiple scans (views from a camera), and registering the scans by computing transformations between the scans. The 3D data may be projected into a 3D space for the transformations and stitching. The scans may be integrated into a common reference frame by applying appropriate transformations to points of each registered scan and projecting each scan into the 3D space.

In one embodiment, registration is performed for adjacent or overlapping intraoral scans (e.g., each successive frame of an intraoral video). In one embodiment, registration is performed using blended scans and/or reduced or cropped scans. Registration algorithms are carried out to register two or more adjacent intraoral scans and/or to register an intraoral scan with an already generated 3D surface, which essentially involves determination of the transformations which align one scan with the other scan and/or with the 3D surface. Registration may involve identifying multiple points in each scan (e.g., point clouds) of a scan pair (or of a scan and the 3D model), surface fitting to the points, and using local searches around points to match points of the two scan (or of the scan and the 3D surface). For example, the intraoral scan application may match points of one scan with the closest points interpolated on the surface of another image, and iteratively minimize the distance between matched points. Other registration techniques may also be used. The intraoral scan application may repeat registration and stitching for all scans of a sequence of intraoral scans and update the 3D surface as the scans are received.

When a scan session is complete (e.g., all scans for an intraoral site or dental site have been captured), the intraoral scan application may generate a virtual 3D model (also referred to as a digital 3D model) of one or more scanned dental sites. The virtual 3D model includes a 3D surface of the one more scanned dental sites, but has a higher degree of accuracy than the 3D surface generated during the scanning process. To generate the virtual 3D model, the intraoral scan application may register and “stitch” or merge together the intraoral scans generated from the intraoral scan session. In one embodiment, registration is performed for adjacent and/or overlapping intraoral scans (e.g., each successive frame of an intraoral video). In one embodiment, registration is performed using blended scans and/or reduced or cropped scans. Registration algorithms may be carried out to register two or more adjacent intraoral scans and/or to register an intraoral scan with a 3D model, which essentially involves determination of the transformations which align one scan with the other scan and/or with the 3D model. Registration may involve identifying multiple points in each scan (e.g., point clouds) of a scan pair (or of a scan and the 3D model), surface fitting to the points, and using local searches around points to match points of the two scans (or of the scan and the 3D model). For example, the intraoral scan application may match points of one scan with the closest points interpolated on the surface of another scan, and iteratively minimize the distance between matched points. Other registration techniques may also be used. The registration and stitching that are performed to generate the 3D model may be more accurate than the registration and stitching that are performed to generate the 3D surface that is shown in real time or near-real time during the scanning process.

The intraoral scan application may repeat registration for all scans of a sequence of intraoral scans to obtain transformations for each scan, to register each scan with the previous one and/or with a common reference frame (e.g., with the 3D model). The Intraoral scan application integrates all scans into a single virtual 3D model by applying the appropriate determined transformations to each of the scans. Each transformation may include rotations about one to three axes and translations within one to three planes.

In one embodiment, the intraoral scan application includes a treatment planner configured to perform treatment planning for orthodontic treatment and/or prosthodontic treatment. The treatment planner may additionally perform dental diagnostics and/or prognostics. The treatment planner in embodiments generates an orthodontic treatment plan, including a 3D model for a final tooth arrangement and 3D models for one or more intermediate tooth arrangements.

The following non-limiting example may help understand the process more fully. A patient who wishes to straighten their teeth may opt for Invisalign® treatment. Invisalign is a process that creates a custom made series of clear aligners specifically for the patient. The clear aligners are worn over the patient's teeth and gradually shift the patient's teeth. A new set of aligners may be worn after a specified period of time (e.g., two weeks) until treatment is complete.

The patient may visit a dental practitioner or orthodontist to begin Invisalign treatment. The dental practitioner may utilize intraoral scanner 100 to scan the patient's teeth in a scanning mode. The dental practitioner may use scanner 100 to capture the patient's teeth segments (e.g., upper arch, lower arch, bite segments) in one or more sets of intraoral scans. The intraoral scan application may register and stitch together the intraoral scans to create a 3D rendering of the scanned segments and present the 3D rendering to the dental practitioner. Once the scans are completed, the dental practitioner may next navigate to the image processing mode, which may generate a virtual 3D model by registering and stitching together the intraoral images. Once an adequate set of 3D renderings and/or virtual 3D model are complete, the 3D renderings and/or 3D models may be saved to the patient profile.

The dental practitioner may then provide input to switch to a planning mode, in which a final tooth arrangement may be determined and one or more intermediate tooth arrangements may be determined. A treatment plan may be generated to provide a progression of treatment stages from the patient's initial tooth arrangement to the target final tooth arrangement, where a separate 3D model is associated with each treatment stage.

Once an adequate set of 3D models is generated, the 3D models may be saved to a patient profile. The dental practitioner may then navigate to a delivery mode to electronically send the completed patient profile to a processing center. The processing center may then generate the custom made series of clear aligners for the patient and deliver the clear aligners to the dental practitioner. The patient would then return to the dental practitioner to receive the first set of clear aligners and verify the clear aligners properly fit onto the patient's teeth.

It is to be understood that the above description is intended to be illustrative, and not restrictive. Many other embodiments will be apparent upon reading and understanding the above description. Although embodiments of the present disclosure have been described with reference to specific example embodiments, it will be recognized that the disclosure is not limited to the embodiments described, but can be practiced with modification and alteration within the spirit and scope of the appended claims. Accordingly, the specification and drawings are to be regarded in an illustrative sense rather than a restrictive sense. The scope of the disclosure should, therefore, be determined with reference to the appended claims, along with the full scope of equivalents to which such claims are entitled.

1. An intraoral scanner comprising:
an elongate wand comprising a probe at a distal end of the elongate wand;
one or more light sources disposed within the elongate wand away from the distal end of the probe, wherein the one or more light sources are configured to generate unpatterned light;
one or more waveguides disposed within the probe, wherein the one or more waveguides are configured to receive the unpatterned light generated by the one or more light sources and to output patterned light from one or more locations at the distal end of the probe; and
one or more image sensors disposed at the distal end of the probe, wherein the one or more image sensors are configured to capture images of intraoral objects illuminated by the patterned light.

2. The intraoral scanner of claim 1, wherein the one or more waveguides comprise one or more pattern projectors at the distal end of the probe, wherein each pattern projector outputs at least a portion of the patterned light.

3. The intraoral scanner of claim 2, wherein each of the one or more pattern projectors comprises a focusing grating coupler.

4. The intraoral scanner of claim 3, wherein at least one focusing grating coupler is configured to output a pattern of spots.

5. The intraoral scanner of claim 4, wherein the pattern of spots comprises four to sixteen spots having a uniform spacing.

6. The intraoral scanner of claim 2, wherein each waveguide of the one or more waveguides comprises a transport region having a first width, a pattern projector region comprising the pattern projector, the pattern projector region having a second width that is greater than the first width, and a tapered region between the transport region and the pattern projector region that transitions from the first width to the second width.

7. The intraoral scanner of claim 2, further comprising:
a microlens array disposed over each of the one or more pattern projectors.

8. The intraoral scanner of claim 1, wherein the one or more waveguides comprise at least one waveguide configured to perform the following:
receive the unpatterned light from a light source of the one or more light sources;
divide the unpatterned light across a plurality of paths, wherein each path of the plurality of paths comprises a distinct pattern generator configured to output a portion of the patterned light.

9. The intraoral scanner of claim 8, wherein each distinct pattern generator comprises a focusing grating coupler.

10. The intraoral scanner of claim 1, wherein the probe comprises a longitudinal axis, wherein the one or more image sensors each have an angle of 45 degrees to 135 degrees to the longitudinal axis, and wherein the patterned light is projected from the one or more locations at the distal end of the probe at one or more angles of 45 degrees to 135 degrees to the longitudinal axis.

11. The intraoral scanner of claim 10, wherein a lateral distance between the one or more locations at the distal end of the probe and the one or more image sensors within a plane defined at least in part by the longitudinal axis of the probe is less than 2 mm.

12. The intraoral scanner of claim 1, wherein the patterned light has an approximately constant overlap over an entire depth of focus of the intraoral scanner.

13. The intraoral scanner of claim 1, wherein all light rays of the patterned light output from a particular location of the one or more locations are approximately parallel to one another.

14. The intraoral scanner of claim 13, wherein first light rays of the patterned light output from a first location of the one or more locations have a first angle relative to a plane defined at least in part by a longitudinal axis of the probe, and wherein second light rays of the patterned light output from a second location of the one or more locations have a second angle relative to the plane defined at least in part by the longitudinal axis.

15. The intraoral scanner of claim 1, wherein the one or more light sources comprise a first light source that outputs first coherent light having a first wavelength and a second light source that outputs second coherent light having a second wavelength, wherein a same waveguide of the one or more waveguides is to transport the first coherent light and the second coherent light to the one or more locations at the distal end of the probe.

16. The intraoral scanner of claim 1, further comprising at least one of a lens, a prism or a grating that couples a light source of the one or more light sources to a waveguide of the one or more waveguides.

17. The intraoral scanner of claim 1, wherein the one or more light sources are directly coupled to the one or more waveguides.

18. The intraoral scanner of claim 1, wherein:
the one or more waveguides comprises a first plurality of waveguides; and
the one or more light sources comprises a plurality of semiconductor lasers.

19. The intraoral scanner of claim 1, further comprising:
a refractive coating on at least one surface of the one or more waveguides at the one or more locations.

20. The intraoral scanner of claim 1, further comprising:
a first plate in the probe, wherein the first plate covers at least a first subset of the one or more image sensors such that returning light from the imaged intraoral objects passes through the first plate to reach the first subset of the one or more image sensors, wherein the first plate comprises at least a first subset of the one or more waveguides.

21. The intraoral scanner of claim 20, further comprising:
a second plate in the probe disposed on the first plate such that the second plate also covers at least the first subset of the one or more image sensors, wherein the second plate comprises at least a second subset of the one or more waveguides, wherein the first subset of the one or more waveguides output the patterned light from a first subset of the one or more locations, and wherein the second subset of the one or more waveguides output the patterned light from a second subset of the one or more locations that do not overlap with the first subset of the one or more locations.

22. The intraoral scanner of claim 20, wherein the first plate has a first orientation relative to a plane defined at least in part by a longitudinal axis of the probe, the intraoral scanner further comprising:
a second plate in the probe, wherein the second plate covers at least a second subset of the one or more image sensors such that returning light from the imaged intraoral objects passes through the second plate to reach the second subset of the one or more image sensors, wherein the second plate comprises at least a second subset of the one or more waveguides, and wherein the second plate has a second orientation relative to the plane defined at least in part by the longitudinal axis of the probe.

23. An intraoral scanner comprising:
an elongate wand comprising a probe at a distal end of the elongate wand;
one or more light sources disposed within the elongate wand, wherein the one or more light sources are configured to generate unpatterned light, wherein the one or more light sources are oriented to project the unpatterned light in a first direction along a length of the elongate wand; and
one or more pattern generating optical elements disposed within the probe, wherein the one or more pattern generating optical elements are configured to receive the unpatterned light generated by the one or more light sources and to output patterned light from one or more locations of the probe.

24. The intraoral scanner of claim 23, wherein the one or more pattern generating optical elements comprise one or more waveguides.

25. The intraoral scanner of claim 24, wherein each of the one or more waveguides comprises one or more focusing grating couplers that generates the patterned light at the one or more locations.

26. The intraoral scanner of claim 23, wherein the one or more locations are at the distal end of the probe.

27. The intraoral scanner of claim 23, wherein the one or more light sources are located away from the distal end of the probe, and wherein the one or more pattern generating optical elements are to output the patterned light in a second direction that is at an angle to the first direction and in a field of view of one or more cameras of the intraoral scanner.

28. The intraoral scanner of claim 23, wherein the one or more pattern generating optical elements comprises a reflector to redirect the unpatterned light from the first direction to the second direction.

29. An intraoral scanner comprising:
one or more lasers disposed within the intraoral scanner away from a distal end of the intraoral scanner, wherein the one or more lasers are configured to generate first unpatterned light having a first wavelength;
one or more waveguides disposed within the probe, wherein the one or more waveguides each comprise a transport region configured to receive the first unpatterned light and transport the unpatterned light to a projection region comprising a focusing grating coupler, wherein the focusing grating coupler is to output patterned light onto an object external to the intraoral scanner; and
one or more image sensors disposed at the distal end of the intraoral scanner, wherein the one or more image sensors are configured to capture images of intraoral objects illuminated by the patterned light.
